SEQUENCE LISTING (29 sequences)

SEQ ID NO: 1 - DNA, 19 bp, Artificial Sequence
  Synthetic oligonucleotide
  misc_feature (5)..(17): n is a, c, g, t or u
gcgannnnnnnnnnnnnuu

SEQ ID NO: 2 - DNA, 19 bp, Artificial Sequence
  Synthetic oligonucleotide
  misc_feature (5)..(17): n is a, c, g, t or u
ccatnnnnnnnnnnnnnuu

SEQ ID NO: 3 - DNA, 17 bp, Artificial Sequence
  Synthetic oligonucleotide
  misc_feature (5)..(17): n is a, c, g, t or u
ccatnnnnnnnnnnnnn

SEQ ID NO: 4 - DNA, 17 bp, Artificial Sequence
  Synthetic oligonucleotide
  misc_feature (5)..(17): n is a, c, g, t or u
gcgannnnnnnnnnnnn

SEQ ID NO: 5 - DNA, 17 bp, Artificial Sequence
  Synthetic oligonucleotide
gttttcccagtcacgac

SEQ ID NO: 6 - DNA, 17 bp, Artificial Sequence
  Synthetic oligonucleotide
caggaaacagctatgac

SEQ ID NO: 7 - DNA, 50 bp, Artificial Sequence
  Synthetic oligonucleotide
  misc_feature (21)..(33): n is a, c, g, t or u
cgacagtaactacacggcgannnnnnnnnnnnngttttcccagtcacgac

SEQ ID NO: 8 - DNA, 50 bp, Artificial Sequence
  Synthetic oligonucleotide
  misc_feature (21)..(33): n is a, c, g, t or u
gtagcaattggcaggtccatnnnnnnnnnnnnncaggaaacagctatgac

SEQ ID NO: 9 - DNA, 57 bp, Artificial Sequence
  Synthetic oligonucleotide
aatgatacggcgaccaccgagatctacacacgtaggccgacagtaactacacggcga

SEQ ID NO: 10 - DNA, 63 bp, Artificial Sequence
  Synthetic oligonucleotide
  misc_feature (25)..(33): n is a, c, g, t or u
caagcagaagacggcatacgagatnnnnnnnnngaccgtcggcgtagcaattggcaggtccat

SEQ ID NO: 11 - DNA, 28 bp, Artificial Sequence
  Synthetic oligonucleotide
acgtaggccgacagtaactacacggcga

SEQ ID NO: 12 - DNA, 30 bp, Artificial Sequence
  Synthetic oligonucleotide
gaccgtcggcgtagcaattggcaggtccat

SEQ ID NO: 13 - DNA, 30 bp, Artificial Sequence
  Synthetic oligonucleotide
atggacctgccaattgctacgccgacggtc

SEQ ID NO: 14 - DNA, 41 bp, Artificial Sequence
  Synthetic oligonucleotide
ctaaatggctgtgagagagctcaggttttcccagtcacgac

SEQ ID NO: 15 - DNA, 41 bp, Artificial Sequence
  Synthetic oligonucleotide
actttatcaatctcgctccaaacccaggaaacagctatgac

SEQ ID NO: 16 - DNA, 61 bp, Artificial Sequence
  Synthetic oligonucleotide
aatgatacggcgaccaccgagatctacacacgtaggcctaaatggctgtgagagagctcag

SEQ ID NO: 17 - DNA, 67 bp, Artificial Sequence
  Synthetic oligonucleotide
  misc_feature (25)..(33): n is a, c, g, t or u
caagcagaagacggcatacgagatnnnnnnnnngaccgtcggcactttatcaatctcgctccaaacc

SEQ ID NO: 18 - DNA, 32 bp, Artificial Sequence
  Synthetic oligonucleotide
acgtaggcctaaatggctgtgagagagctcag

SEQ ID NO: 19 - DNA, 34 bp, Artificial Sequence
  Synthetic oligonucleotide
gaccgtcggcactttatcaatctcgctccaaacc

SEQ ID NO: 20 - DNA, 34 bp, Artificial Sequence
  Synthetic oligonucleotide
ggtttggagcgagattgataaagtgccgacggtc

SEQ ID NO: 21 - DNA, 20 bp, Artificial Sequence
  Synthetic oligonucleotide
cgacagtaactacacggcga

SEQ ID NO: 22 - DNA, 17 bp, Artificial Sequence
  Synthetic oligonucleotide
gttttcccagtcacgac

SEQ ID NO: 23 - DNA, 20 bp, Artificial Sequence
  Synthetic oligonucleotide
gtagcaattggcaggtccat

SEQ ID NO: 24 - DNA, 17 bp, Artificial Sequence
  Synthetic oligonucleotide
caggaaacagctatgac

SEQ ID NO: 25 - DNA, 17 bp, Artificial Sequence
  Synthetic oligonucleotide
gtcgtgactgggaaaac

SEQ ID NO: 26 - DNA, 13 bp, Artificial Sequence
  Synthetic oligonucleotide
  misc_feature (1)..(13): n is a, c, g, t or u
nnnnnnnnnnnnn

SEQ ID NO: 27 - DNA, 13 bp, Artificial Sequence
  Synthetic oligonucleotide
attcggcggatat

SEQ ID NO: 28 - DNA, 59 bp, Artificial Sequence
  Synthetic oligonucleotide
ggttcgccgcggcgacgaagaaaccgaaaaacgcgttgaacacgacattgttcgcgaag

SEQ ID NO: 29 - DNA, 67 bp, Artificial Sequence
  Synthetic oligonucleotide
catgacaaaattcgtttattaattcgcattgacattgacattcgccgcaaactgggcgattaacaaa

(19) United States
(12) Patent Application Publication
(10) Pub. No.: US 2018/0320166 A1
(43) Pub. Date: Nov. 8, 2018

(54) MULTIPLEX PAIRWISE ASSEMBLY OF DNA OLIGONUCLEOTIDES

(71) Applicant: UNIVERSITY OF WASHINGTON, Seattle, WA (US)

(72) Inventors: Marc Joseph LAJOIE, Seattle, WA (US); Jason Chesler KLEIN, Seattle, WA (US); Jerrod Joseph SCHWARTZ, San Francisco, CA (US); David BAKER, Seattle, WA (US); Jay Ashok SHENDURE, Seattle, WA (US); Lance Joseph STEWART, Bainbridge Island, WA (US)

(73) Assignee: University of Washington, Seattle, WA (US)

(21) Appl. No.: 15/765,045

(22) PCT Filed: Oct. 1, 2016
(86) PCT No.: PCT/US2016/055078

§ 371 Date: Mar. 30, 2018

Related U.S. Application Data
(60) Provisional application No. 62/235,974, filed on Oct. 1, 2015.

(51) Int. Cl. C12N 15/10

(52) U.S. Cl. CPC C12N 15/1031

(57) ABSTRACT

The present invention provides methods for multiplex assembly of oligonucleotides.

### CROSS REFERENCE

This application is related to U.S. provisional patent application, Ser. No. 62/235,974, filed Oct. 1, 2015, the disclosure of which is incorporated by reference herein in its entirety.

### STATEMENT REGARDING FEDERALLY SPONSORED RESEARCH

This invention was made with U.S. government support under Department of Energy-Lawrence Berkeley National Laboratory-Joint Genome Institute award number DE-AC02-05CH11231, and National Institutes of Health (NIH) award number 1R21CA160080. The U.S. Government has certain rights in the invention.

### SEQUENCE LISTING

The sequence listing submitted herewith, entitled “16-1242-PCT_SequenceListing_ST25.txt” and 7 kb in size, is incorporated by reference in its entirety.

### BACKGROUND

Traditionally, DNA has been synthesized by solid-phase phosphoramidite chemistry. Column-based synthesis generates up to 200-mers with error rates of about 1 in 200 nucleotides and yields of 10 to 100 nmol per product. Column based DNA synthesis is limited in throughput to 384-wellplates, and oligonucleotides cost from $0.05 to $1.00/base-pairs (bp) depending on length and yield. The commercialization of inkjet-based printing of nucleotides with phosphoramidite chemistries (e.g., Agilent) and semiconductor-based electrochemical acid production arrays (e.g., CustomArray) have increased throughput and decreased the cost of oligonucleotide synthesis. These oligonucleotides range from $0.00001-0.001/bp in cost, depending on length, scale and platform. However, these platforms are limited by short synthesis lengths, high synthesis error rates, low yield and the challenges of assembling long constructs from complex pools.

Many methods have recently addressed the high error rates of array-synthesized oligonucleotides, with a trade-off between cost and fidelity. Low-cost methods include proteins such as MutS, polymerases and other proteins that bind and cut heteroduplexes. However, as these methods rely on identifying mismatches and require the majority of sequences to be identical, they are not always compatible with complex libraries and therefore must be performed after individual gene assemblies. Furthermore, as these methods retain error rates as high as 1 per 1000 nucleotides, further screening is required to confirm the correct sequence. More recent methods such as Dial-Out PCR rely on DNA sequencing followed by retrieval of sequence-verified constructs, achieving error rates as low as 10⁻⁷. While these methods can work on complex oligonucleotide pools and yield very low error rates, they are costly, time-intensive and do not always recover targeted molecules.

Despite their high error rates, inexpensive oligonucleotide pools cleaved from microarrays have recently enabled high-throughput analysis of promoter and enhancer function, providing novel insight into the vocabulary of these regulatory elements. They have also been used in deciphering the role of genetic variants in protein function. However, these studies were all limited by short synthesis lengths—about 160 bp for CustomArray and 230 bp for Agilent.

Short synthesis lengths and high error rates present bottlenecks to the use of array-derived oligonucleotides for both functional assays and gene assembly. Described herein is a method to assemble thousands of array-derived oligonucleotides into targets approaching length estimates of cis-regulatory elements and protein domains. Compared to existing methods, the methods described here do not limit sequence space by using restriction enzymes, are high throughput, and offer an efficient way to retrieve error-free assemblies.

### SUMMARY OF THE INVENTION

In a first aspect, the present invention provides a method for assembly of one or more double-stranded polynucleotides, the method comprising: (a) amplifying a first plurality of single-stranded overlapping oligonucleotides, wherein the first plurality of single-stranded overlapping oligonucleotides comprises: (i) overlapping regions with homology capable of annealing to produce one or more double-stranded polynucleotides, and (ii) at least one common primer binding site in each single-stranded overlapping oligonucleotide; (b) assembling one or more double-stranded polynucleotides, wherein the assembling comprises denaturing, annealing and extending the first plurality of single-stranded overlapping oligonucleotides to generate the one or more double-stranded polynucleotides.

The inventors have surprisingly discovered that methods of the present invention provide high-throughput, multiplex assembly of thousands of polynucleotides between approximately 200-400 or more nucleotides in length. Furthermore, the methods of the invention provide efficient way to retrieve error-free assemblies of the thousands of polynucleotides. These findings can provide methods for both complex library generation and gene synthesis. For example, creating a library of 3,118 such 200 bp polynucleotides would be ˜38-fold less expensive than column-based synthesis methods (˜0.84 USD/target). The methods of the invention can be utilized to synthesize polynucleotide libraries at an unprecedented cost allowing researchers to address questions using precisely designed sequences rather than relying on biased mutagenesis methods. Moreover, the methods described herein can be used for gene synthesis, gene regulation, protein function and directed evolution, all of which have contributed to novel pharmaceuticals and a better understanding of genome organization. Finally, increasing the length of polynucleotide assemblies that can be produced with low-cost, high complexity DNA synthesis will provide new opportunities for protein design and synthetic biology.

In some embodiments, the method further comprises: (c) tagging the one or more double-stranded polynucleotides, wherein the tagging comprises amplifying the one or more double-stranded oligonucleotides using a pair of tagging primers to generate one or more tagged double-stranded polynucleotides, wherein each tagging primer in the pair of tagging primers comprises: (i) a first segment comprising a unique flanking sequence, and (ii) a second segment comprising a seed sequence; (d) sequencing the one or more tagged double-stranded polynucleotides, wherein the sequencing comprises binding of the seed sequence to a sequencing platform and performing a sequencing reaction to identify one or more sequence verified polynucleotides; and (e) retrieving the one or more sequence verified polynucleotides, wherein the retrieving comprises base-pairing a complementary primer to the first segment of at least one tagging primer in the one or more sequence verified polynucleotides and, under conditions suitable and in the presence of suitable reagents, amplifying the sequence verified polynucleotides to produce one or more verified polynucleotides; or (c) phenotypic selection of functional polypeptides, wherein the phenoytypic selection comprises of one or more of yeast display, phage display, mRNA display, ribosome display, mammalian cell display, bacterial cell display, emulsion-based protein selection, functional complementation of a portion of a genome, or other selection methods known to experts in the field of polypeptideevolution.

In another embodiment, the method further comprises step-wise assembly of two or more of the double-stranded or verified polynucleotides into an assembled polynucleotide product, wherein the two or more double-stranded or verified polynucleotides have overlapping regions with homology capable of annealing and at least one common primer binding site in each of the double-stranded or verified polynucleotides; and (f) combining the two more double-stranded or verified polynucleotides under conditions suitable for annealing the overlapping regions with homology and in the presence of suitable reagents for assembling an initial desired polynucleotide product by extension of the double-stranded or verified polynucleotides to produce the initial desired polynucleotide product; and (g) combining the initial desired polynucleotide product and a next double-stranded or verified polynucleotide, wherein the initial desired polynucleotide product and the next double-stranded or verified polynucleotide have overlapping regions with homology capable of annealing and at least one common primer binding site in each of the double-stranded or verified polynucleotides, and assembling the initial desired polynucleotide product and the next double-stranded or verified polynucleotide in the presence of suitable reagents for assembling the assembled polynucleotide product by extension of the initial desired polynucleotide product and the next double-stranded or verified polynucleotide; and (h) reiteratively repeating (g) to step-wise add additional next double-stranded or verified polynucleotides to the initial desired polynucleotide product to produce the assembled polynucleotide product.

In yet another embodiment, the method further comprises hierarchical assembly of two or more of the double-stranded or verified polynucleotides into an assembled polynucleotide product, wherein the two or more double-stranded or verified polynucleotides have overlapping regions with homology capable of annealing and at least one common primer binding site in each of the double-stranded or verified polynucleotides; and (f) combining the two double-stranded or verified polynucleotides under conditions suitable for annealing the overlapping regions with homology and in the presence of suitable reagents for assembling a first desired polynucleotide product by extension of the double-stranded or verified polynucleotides to produce the first desired polynucleotide product; and (g) repeating (f) with another two double-stranded or verified polynucleotides to produce a second desired polynucleotide product; (e) combining the first desired polynucleotide product and the second desired polynucleotide product, wherein the first desired polynucleotide product and the second desired polynucleotide product have overlapping regions with homology capable of annealing and at least one common primer binding site in each of the first and the second desired polynucleotide products, and assembling the first desired polynucleotide product and the second desired polynucleotide product in the presence of suitable reagents for assembling the assembled polynucleotide product by extension of the first desired polynucleotide product and the second desired polynucleotide product; and (h) repeating (f), (g) and (e) to hierarchically assemble pairs of desired polynucleotides to produce the assembled polynucleotide product.

### BRIEF DESCRIPTION OF THE DRAWINGS

The disclosed exemplary aspects have other advantages and features which will be more readily apparent from the detailed description, the appended claims, and the accompanying figures. A brief description of the drawings is below.

FIG. 1 shows an overview of multiplex pairwise assembly. A total of 2,271 oligonucleotide targets were separated into 10 sets of 131-250 oligonucleotides. Each oligonucleotide was split into A and B fragments with overlapping sequences providing >56° C. melting temperature (Tm) for PCR-mediated assembly. All oligonucleotides were cleaved off the array into one tube. Each sub-pool was then amplified with one common primer and one uracil-containing pool-specific primer. The uracil-containing pool-specific primer was then removed with Uracil Specific Excision Reagent (USER™) followed by New England BioLabs End Repair kit. During PCR assembly, corresponding sub-pools were allowed to anneal and extend through 5 cycles of PCR, before adding a set of common, outer primers for amplification. During PCR assembly, M13F and M13R sequences can be introduced to the polynucleotide products in order to allow for Dial-Out Tagging and retrieval of sequence-verified polynucleotide products. Up to 252-mers were assembled from 160-mer CustomArray oligonucleotides.

FIG. 2 shows a pipeline for generation of static tag library. First, 1.2 million random 13-mers (5′-NNNNNNNNNNNNN-3′; SEQ ID NO:26) were generated, and screened for no homoguanine or homocytosine stretches >5 bp (5′-ATTCGGCGGATAT-3′; SEQ ID NO:27), no homoadenine or homothymine stretches >8 bp and GC content between 45% and 65%. The 13-mers were also screened for <90% nucleotide identity in the last 10 bp, which generated a set of 7,411 13-mers. From this set of 7,411 sequences, every pairwise Gibbs free energy was calculated, and the maximum number of sequences such that no two members had a dG≤-9 kcal/mol were identified. This left a set of 4,637 sequences, which were split into a set of 2,318 forward tags and 2,319 reverse tags.

FIG. 3A shows a uniformity plot of error-free array-derived oligonucleotides by rank-ordered percentile for all 2,271 oligonucleotide targets assembled in sets of 131-250.

FIG. 3B shows the number and size of oligonucleotide targets, and error-free yield for each set of oligonucleotides assembled in sets of 131-250.

FIG. 3C shows the percent yield of assemblies when assembling oligonucleotide targets in sets of 131-250. Each oligonucleotide target is placed into a bin based on the limiting oligonucleotide count, which is the number of error-free reads out of 1.2 million that are limiting for its corresponding oligonucleotide target. The percent yield of assemblies is the percentage of oligonucleotide targets in that bin with at least one perfect assembly.

FIG. 3D shows the percentage of perfect, mismatch only, small indel (<5 bp), large indel (≥5 bp), truncations and unmapped reads for all oligonucleotides when assembled in sets of 131-250.

FIG. 3E shows the percentage of perfect, mismatch only, small indel (<5 bp), large indel (≥5 bp), chimeras, truncations and unmapped reads for each assembled library set when assembled in sets of 131-250.

FIG. 3F shows the uniformity of each set of oligonucleotide targets (sets 1-9 are between 131-250 oligonucleotide targets and set 10 has 131 oligonucleotide targets).

FIG. 4A shows the effect of complexity on assembly performance and the percentage of oligonucleotide targets with at least one error-free assembly for each level of complexity.

FIG. 4B shows the effect of complexity on assembly performance and the yield (number of oligonucleotide targets with at least one perfect read) versus complexity. Red bars show the total number of oligonucleotide targets with error free assemblies at each level of complexity. Black bars show the number of oligonucleotide targets from the corresponding sets with error-free assemblies, which were individually assembled in sets of complexity ranging from 131-250.

FIG. 4C shows the effect of complexity on assembly performance and that each oligonucleotide target is placed into a bin based on the limiting oligonucleotide count, which is the number of error-free reads (out of 1.2 million), that are limiting for its corresponding oligonucleotide target. The percent yield of assemblies is the percentage of oligonucleotide targets in that bin with at least one perfect assembly.

FIG. 4D shows the effect of complexity on assembly performance and the percentage of perfect, mismatch only, small indels (<5 bp), large indels (≥5 bp), chimeras, truncations and unmapped reads in sets of increasing complexity.

FIG. 4E shows the effect of complexity on assembly performance and the uniformity of each set of oligonucleotide targets.

FIG. 5A shows the error correction of assembled constructs and the per-base accuracy of assembled constructs in black and their corresponding oligonucleotides in red and blue. Increased accuracy is seen at both priming sites and the overlap region.

FIG. 5B shows the error correction of assembled constructs and the bar graphs for the percentage of tags identified on only one, two, three, four or at least five different molecules in the sequenced library. Orange (pool 2) and purple bars (pool 6) are two different assembly sets, each with 250 oligonucleotide targets

FIG. 5C shows the error correction of assembled constructs and the percentage of aligning reads that contain no errors for each of the 25 retrieved assemblies.

FIG. 6A shows the percentage of perfect, mismatch only, small indels (<5 bp), large indels (≥5 bp), chimeras, truncations, and unmapped reads for assemblies using one or two unique primers for initial amplification of oligonucleotides, for two independent sub pools when comparing one versus two unique primers per oligonucleotide pool. Pools of oligonucleotides were amplified off the array using either one unique primer (Uracil-containing A/B fragment primer) and one common primer (YF/YR), or two unique primers (Uracil-containing A/B fragment primer and A/B fragment unique F/R) (Table 1). Each pool was then assemble and sequenced to 115,000 reads.

FIG. 6B shows the uniformity for one sub pool with one or two unique primers when comparing one versus two unique primers per oligonucleotide pool.

FIG. 7A shows a representative Sanger trace (SEQ ID NO:28) for 22/25 retrieval reactions for dial-out PCR retrieval.

FIG. 7B shows a representative Sanger trace (SEQ ID NO:29) for 3/25 retrieval reactions for dial-out PCR retrieval.

FIG. 8A shows oligonucleotide uniformity across 10,000 oligonucleotides corresponding to 10 sub-pools of oligonucleotide targets for assembly without duplicated oligonucleotides.

FIG. 8B shows assembly yield of sets of 500 oligonucleotide targets for assembly without duplicated oligonucleotides.

FIG. 8C shows aggregate data for assembly without duplicated oligonucleotides from all pools of 500. Each oligonucleotide target is placed into a bin based on the limiting oligonucleotide count, which is the number of error-free reads (out of 525K), that are limiting for its corresponding oligonucleotide target. Percent yield of assemblies is the percentage of oligonucleotide targets in that bin with ≥1 perfect assembly.

FIG. 8D shows aggregate data for assembly without duplicated oligonucleotides from all pools of 2,000. Each oligonucleotide target is placed into a bin based on the limiting oligonucleotide count, which is the number of error-free reads (out of 525,000), that are limiting for its corresponding oligonucleotide target. Percent yield of assemblies is the percentage of oligonucleotide targets in that bin with ≥1 perfect assembly

FIG. 9 shows yield versus oligonucleotide target length. After assembly, oligonucleotide targets were binned according to their target size. Black bars show the % of oligonucleotide targets assembled with at least one error-free yield in individual sub pools of 131-250. Red bars show the same breakdown for assembly in one pool of 2,271 oligonucleotide targets.

FIG. 10 shows the uniformity plots of each set 1 and set 9 of oligonucleotide targets when performed with a higher quality, higher uniformity of input oligonucleotides from Twist compared to previous input oligonucleotides from CustomArray.

FIG. 11 shows a uniformity plot of smaller sets of longer oligonucleotides (230 bp sequences) from a different vendor (Agilent), resulted in assembly of greater than 90% of 393 bp target sequences.

FIG. 12 shows an overview of hierarchical multiplex pairwise assembly.

FIG. 13 shows a DNA gel demonstrating hierarchical multiplex pairwise assembly.

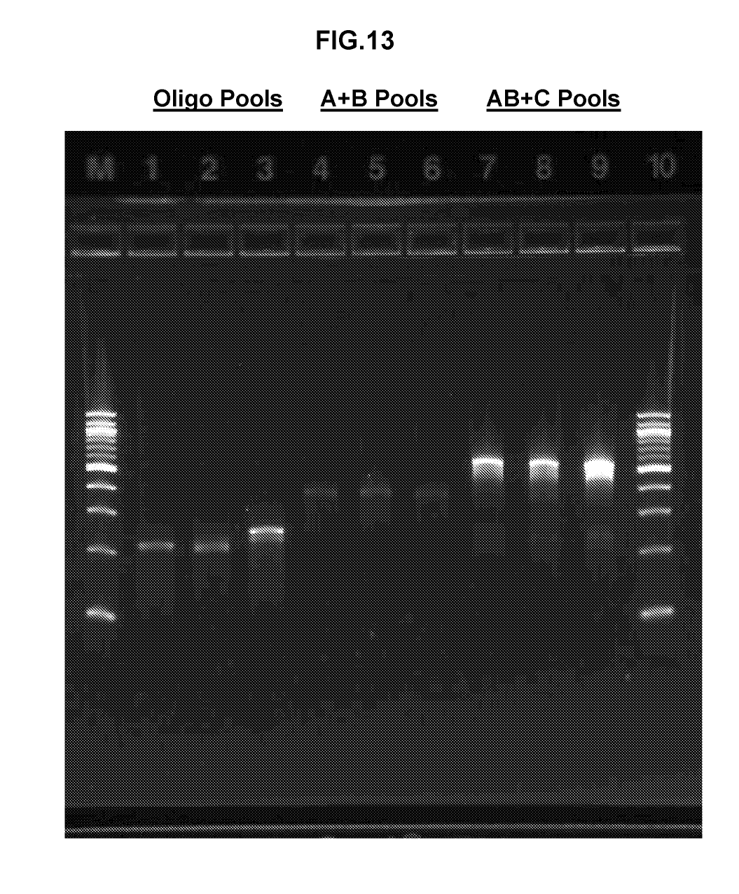

FIG. 14 shows a uniformity plot of a hierarchical multiplex pairwise assembly.

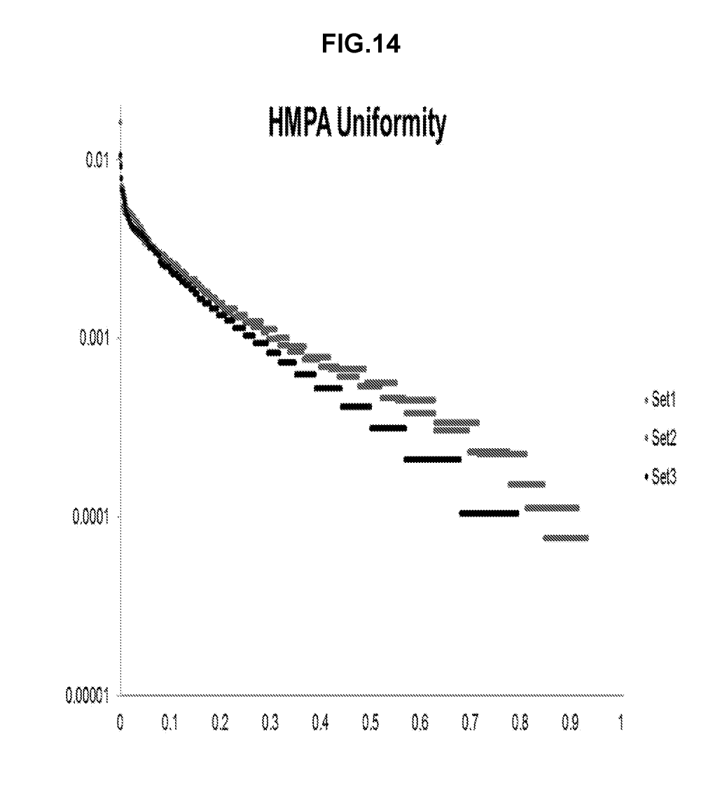

FIG. 15 demonstrates increased adapter cleavage efficiency using USER™ cleavage with additional uracils for adapter cleavage.

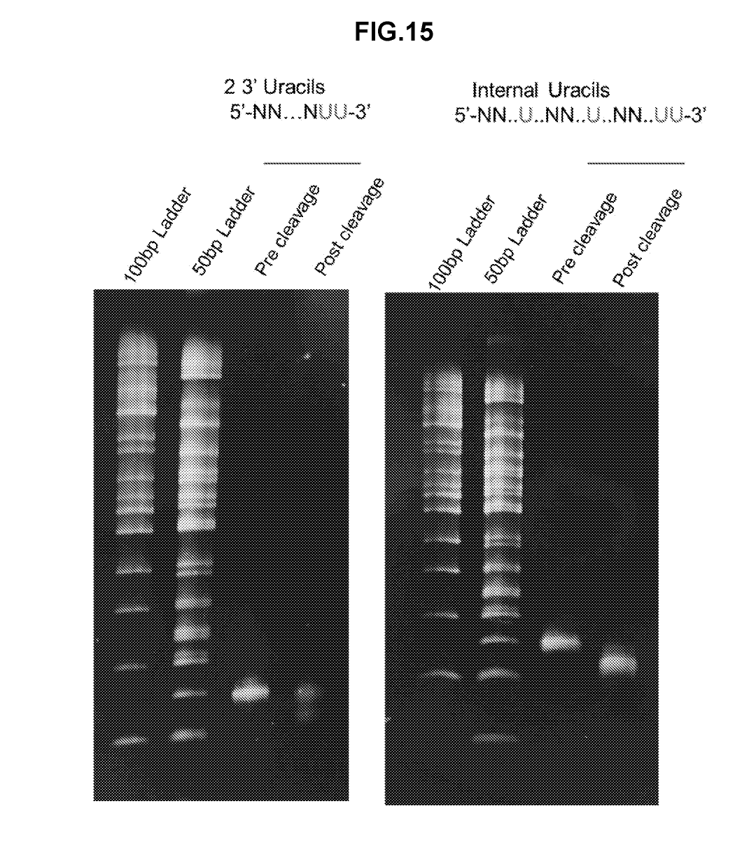

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (RI. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

Terms used in the claims and specification are defined as set forth below unless otherwise specified. In the case of direct conflict with a term used in a parent provisional patent application, the term used in the instant specification shall control.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of skill in the art, such as the Oxford Dictionary of Biochemistry and Molecular Biology (Ed. Anthony Smith, Oxford University Press, Oxford, 2004).

As used herein, the singular forms “a”, “an” and “the” include plural referents unless the context clearly dictates otherwise. “And” as used herein is interchangeably used with “or” unless expressly stated otherwise.

The terms “nucleic acid,” “polynucleotide” and “oligonucleotide” are used interchangeably and refer to deoxyribonucleotides or ribonucleotides or modified forms of either type of nucleotides, and polymers thereof in either single- or double-stranded form. The terms should be understood to include equivalents, analogs of either RNA or DNA made from nucleotide analogs and as applicable to the embodiment being described, single stranded or double stranded polynucleotides. In certain embodiments, an oligonucleotide may be chemically synthesized.

All embodiments disclosed herein can be used in combination unless the context clearly dictates otherwise.

In a first aspect, the present invention provides a method for assembly of one or more double-stranded polynucleotides, the method comprising: (a) amplifying a first plurality of single-stranded overlapping oligonucleotides, wherein the first plurality of single-stranded overlapping oligonucleotides comprises: (i) overlapping regions with homology capable of annealing to produce one or more double-stranded polynucleotides, and (ii) at least one common primer binding site in each single-stranded overlapping oligonucleotide; (b) assembling one or more double-stranded polynucleotides, wherein the assembling comprises denaturing, annealing and extending the first plurality of single-stranded overlapping oligonucleotides to generate the one or more double-stranded polynucleotides.

In some embodiments, the first plurality of single-stranded overlapping oligonucleotides can be derived from an array. In such embodiments, the oligonucleotides may be obtained from a commercial source. For example, the oligonucleotides may be from arrays that are constructed, custom ordered or purchased from a commercial vendor. Such vendors include, but are not limited to, Agilent, Affymetrix, CustomArray, Nimblegen, MycroArray, LC Sciences and Twist. Single-stranded oligonucleotides are typically synthesized in situ on a common support wherein each oligonucleotide is synthesized on a separate spot on the substrate. In an embodiment, oligonucleotides can be of any length, but are typically 10-400 bases long or loner. For example, oligonucleotides may be from 10 to about 300 nucleotides, from 20 to about 400 nucleotides, from 30 to about 500 nucleotides, from 40 to about 600 nucleotides, or more than about 600 nucleotides long. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390 400, 410, 420, 430, 440, 450, 460, 470, 480, 490, 500, 510, 520, 530, 540, 550, 560, 570, 580, 590 and 600 nucleotides in length are contemplated. Oligonucleotides from such an array may be covalently attached to the surface or deposited on the surface. Various methods of array construction are known in the art (for example, maskless array synthesizers, light directed methods utilizing masks, flow channel methods, or spotting methods).

In some embodiments, the plurality of single-stranded oligonucleotides can be two, three, four, 5 or more, 10 or more, 20 or more, 50 or more, 100 or more, 250 or more, 500 or more, 1,000 or more, 1,500 or more, 2,000 or more, or 2,500 or more oligonucleotides. For example, a plurality can be approximately 2-100, 100-250, approximately 250-450, approximately 450-700, approximately 700-950, approximately 950-1,200, approximately 1,200-1,450, approximately 1,450-1,675, approximately 1675-1800, approximately 1,800-2,025, or approximately 2,025-2,275 oligonucleotides. More specifically, a plurality can be 250, or 462, or 712, or 962, or 1212, or 1452, or 1674, or 1805, or 2021 or 2271 oligonucleotides.

The oligonucleotides and/or polynucleotides used and generated in the methods described herein can be predefined or have desired sequences, meaning that the sequences of the oligonucleotides and/or polynucleotides are known and chosen before synthesis or assembly of the oligonucleotides and/or polynucleotides. In some embodiments, the methods described herein use oligonucleotides and/or polynucleotides with sequences determined based on the sequence of the final assembled polynucleotides products to be synthesized. It should be appreciated that different oligonucleotides may be designed to have different lengths. In some embodiments, the sequence of the assembled polynucleotide product may be divided up into a plurality of shorter oligonucleotide sequences that can be assembled step-wise, hierarchically and/or in parallel into a single or a plurality of desired or assembled polynucleotide products using the methods described herein. In certain embodiments, the predefined sequence of each of the oligonucleotides in the first plurality of single-stranded overlapping oligonucleotides further comprises an adaptor sequence. In some embodiments, the adaptor sequence can comprise a degenerate sequence that is a completely degenerative sequence or a partially degenerate sequence.

In certain embodiments, the adaptor sequence may be of any suitable length. In some embodiments, the adaptor sequence is between approximately 5 to 30, 5 to 25, 5 to 20, 5 to 15, 5 to 10, 10 to 30, 10 to 25, 10 to 20, 10 to 15, 15 to 30, 15 to 25, 15 to 20, 20 to 30, 20 to 25, 25 to 30 or more than 30 nucleotides in length. In other embodiments, the adaptor sequence is approximately 5, 6, 7, 8, 9, 10, 11, 12, 13, 14, 15, 16, 17, 18, 19, 20, 21, 22, 23, 24, 25, 26, 27, 28, 29, 30 or more than 30 nucleotides in length. In other embodiments, the adaptor sequence may be up to and approximately 100 or more nucleotides in length. Regardless of its length, the adaptor sequence may include a completely degenerate sequence, a partially degenerate sequence, or a known, non-degenerate sequence. In certain embodiments, the adaptor sequence may be a completely degenerate sequence. For example, an adaptor sequence can comprise a sequence that is 13 nucleotides in length (13-mer) and may have a completely degenerate sequence 5′-NNNNNNNNNNNNN-3′ (SEQ ID NO:26), wherein each N may be any natural or non-natural nucleotides. Although a 13-mer is used as an example, it is understood that the completely degenerate sequence may be of any suitable length as discussed above. In other embodiments, the adaptor sequence may be a partially degenerate sequence interspersed with constant bases. For example, in one embodiment, an adaptor may be 20 nucleotides in length (20-mer) having 15 degenerate nucleotides interspersed with five fixed or constant nucleic acids. In other embodiments, a partially degenerate sequence may include a plurality of constant nucleic acids that are designed to contain a particular CG bias or percentage (e.g., under 40% CG, 40-45% CG, 45-50% CG, 50-55% CG, 55-60% CG, or over 60% CG). Although a 20-mer is used as an example, it is understood that the partially degenerate sequence may be of any suitable length as discussed above. Further, the portions of the partially degenerate sequence that are degenerate or fixed may be determined or designed to be any length or portion thereof, and in any suitable combination. In other embodiments, the oligonucleotides may be tagged with a set of known, non-degenerate adaptor sequences. The set of known, non-degenerate adaptor sequences may be part of a unique flanking sequence used as identification tags as described further below. The unique flanking sequences may be designed such that each known adaptor sequence is different for each member.

In some embodiments, the oligonucleotides or polynucleotides can be amplified to obtain a larger quantity of oligonucleotides or polynucleotides for additional or downstream steps. Polymerase Chain Reaction (PCR) is a DNA amplification method in molecular biology that is routinely carried out by those skilled in the art, and can be used to amplify a single copy or a few copies of a piece of DNA (i.e., an oligonucleotide or polynucleotide) across several orders of magnitude, generating thousands to millions of copies of a particular DNA sequence. PCR relies on thermal cycling, consisting of cycles of repeated heating and cooling of the reaction for DNA melting (i.e., denaturing) and enzymatic replication of the DNA. Primers containing sequences complementary to the target region along with a DNA polymerase, are key components to enable selective and repeated amplification. As PCR progresses, the DNA generated is itself used as a template for replication, setting in motion a chain reaction in which the DNA template is exponentially amplified. Typically, PCR uses a heat-stable DNA polymerase, examples include, but are not limited to, KAPA HIFI™, Taq (a heat-stable DNA polymerase from the bacterium Thermus aquaticus) and Pfu (a thermophilic DNA polymerase with a 3′ to 5′ exonuclease/proofreading activity from Pyrococcus furiosus). Usually, PCR consists of a series of 20-40 repeated temperature changes (i.e., cycles), with each cycle (denaturing, annealing and extending) commonly consisting of 2-3 discrete temperature steps in a solution that comprises a polymerase, primers and dNTPs.

In another embodiment, the oligonucleotides or polynucleotides can include a predefined oligonucleotide assembly sequence flanked by 5′ and 3′ sequences. The predefined oligonucleotide assembly sequence is designed for incorporation into an assembled oligonucleotide or desired polynucleotide product. The flanking sequences are designed for use as adaptors for amplification, tagging or retrieval and are not intended to be incorporated into the assembled oligonucleotide or desired polynucleotide product. The flanking adaptor, amplification, tagging or retrieval sequences may be used as universal primer or common primer or set specific primer sequences to amplify a plurality of different assembly oligonucleotides that share the same amplification sequences, but have different central assembly sequences. In some embodiments, the flanking sequences are removed after amplification to produce an oligonucleotide that contains only the assembly sequence.

In certain embodiments, the oligonucleotides or polynucleotides comprise at least one uracil-containing primer region. In some embodiments, the uracil residue is at the end of an oligonucleotide. In other embodiments, the uracil residue is internal. In yet other embodiments, the uracil-containing primer region contains two consecutive uracil residues. In some embodiments, uracil DNA glycosylase (UDG) may be used to hydrolyze a uracil-glycosidic bond in an oligonucleotide thereby removing uracil and creating an alkali-sensitive a basic site in the DNA which can be subsequently hydrolyzed by endonuclease, heat or alkali treatment. For example, the uracil-containing primer regions are removed from the oligonucleotides by contacting the oligonucleotides with uracil DNA glycosylase and a DNA glycosylase-lyase endonuclease VIII to generate a single nucleotide gap at the location of a uracil.

As used herein, a primer or primer pair refers to an oligonucleotide pair (i.e, a forward and reverse primer), either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase. In certain embodiments, a universal primer or universal primer binding site means that a sequence used to amplify the oligonucleotide is universal to all oligonucleotides such that all such oligonucleotides can be amplified using a single set of universal primers. In certain embodiments, individual or unique primers are specific to each oligonucleotide, and have binding sites on either the 5′ end or the 3′ end or both. In some embodiments, primers/primer binding site may be designed to be temporary. For example, temporary primers may be removed by chemical, light based or enzymatic cleavage. For example, primers/primer binding sites may be designed to include a restriction endonuclease cleavage site or a uracil residue. In an exemplary embodiment, a primer/primer binding site contains at least one uracil residue, which can be removed by contacting the oligonucleotides with uracil DNA glycosylase (UDG) and a DNA glycosylase-lyase endonuclease VIII to generate a single nucleotide gap at the location of a uracil.

In yet another embodiment, the oligonucleotides and/or polynucleotides contain overlapping regions of homology that are capable of annealing and the overlapping regions have a melting temperature (Tm) that is greater than 56° C. The oligonucleotides can include one or more oligonucleotide pairs with overlapping identical sequences, one or more oligonucleotide pairs with overlapping complementary sequences, or a combination thereof. Oligonucleotides and/or polynucleotides being assembled are designed to have overlapping regions with homology capable of annealing (i.e., complementary sequences). In some embodiments, the oligonucleotides and/or polynucleotides are double-stranded DNA. The presence of overlapping regions with homology capable of annealing (complementary sequences) on two DNA fragments promotes the assembly of the oligonucleotides and/or polynucleotides. Overlapping sequences may be of any suitable length. For example, overlapping sequences may encompass the entire length of one or more polynucleotides used in an assembly reaction. Overlapping sequences may be between about 5 and about 500 oligonucleotides long. For example, between about 10 and 100, between about 10 and 75, between about 10 and 50 nucleotides. Or about 20, about 25, about 30, about 35, about 45, about 50, about 55, about 60, about 65, about 70, about 75, about 80, about 85, about 90, about 95 or about 100 nucleotides long. However, shorter, longer, or intermediate overlapping lengths may be used. It should be appreciated that overlaps between different polynucleotides used in an assembly reaction may have different lengths. More specifically, each target polynucleotide can be fragmented into two pieces (e.g. A and B) using a custom python script that determines overlaps with the least chance of cross-hybridization. Briefly, the following procedure was automated using python: bases for the overlap region were dynamically added starting from the midpoint-7 position until the melting temperature was >56° C. The overlap region was then checked against all sequences in a set of oligonucleotides and accepted if <15 consecutive bases aligned to any other sequence in the set. To quickly evaluate alignments against all sequences in a given set, a simple sliding algorithm was utilized, which scores the longest consecutive alignments. If the overlap sequence failed these conditions, up to 6 codons were swapped out at random within this sequence region, and if the melting temperature was still >56° C., the alignment step was repeated. If conditions still were not met, the starting position for the overlap region was shifted and the procedure was repeated. A window of 6 bases around the starting position was explored.

In an embodiment, oligonucleotides and/or polynucleotides can be assembled in a polymerase-mediated assembly reaction from one or more oligonucleotides and/or polynucleotides that are combined and extended in one or more rounds of polymerase-mediated extensions. In some embodiments, the oligonucleotides and/or polynucleotides to be assembled may be amplification products (e.g., PCR products). In other embodiments, assembly of the one or more double-stranded oligonucleotides comprises denaturing, annealing and extending the oligonucleotides and/or polynucleotides. Polymerase-based assembly techniques may involve one or more suitable polymerase enzymes that can catalyze a template-based extension of an oligonucleotide in a 5′ to 3′ direction in the presence of suitable nucleotides and an annealed template. A polymerase may be thermostable. A polymerase may be obtained from recombinant or natural sources. In some embodiments, a thermostable polymerase from a thermophilic organism may be used. In some embodiments, a polymerase may have no, or little, proofreading activity. Examples of thermostable DNA polymerases include, but are not limited to: KAPA HIFI™, Taq (a heat-stable DNA polymerase from the bacterium Thermus aquaticus); Pfu (a thermophilic DNA polymerase with a 3′ to 5′ exonuclease/proofreading activity from Pyrococcus furiosus); VENTR® DNA Polymerase and VENT® (exo-) DNA Polymerase (thermophilic DNA polymerases with or without a 3′ to 5′ exonuclease/proofreading activity from Thermococcus litoralis; also known as Tli polymerase); Deep VENTR® DNA Polymerase and Deep VENTR® (exo-) DNA Polymerase (thermophilic DNA polymerases with or without a 3′ to 5′ exonuclease and/or proofreading activity from Pyrococcus species GB-D; available from New England Biolabs); KOD HiFi (a recombinant Thermococcus kodakaraensis KODI DNA polymerase with a 3′ to 5′ exonuclease/proofreading activity, available from Novagen); BIO-X-ACT (a mix of polymerases that possesses 5′ to 3′ DNA polymerase activity and 3′ to 5′ proofreading activity); Klenow Fragment (an N-terminal truncation of E. coli DNA Polymerase I which retains polymerase activity, but has lost the 5′ to 3′ exonuclease activity, available from, for example, Promega and NEB); SEQUENASE™ (T7 DNA polymerase deficient in 3′ to 5′ exonuclease activity); Phi29 (bacteriophage 29 DNA polymerase, may be used for rolling circle amplification, for example, in a TEMPLIPHI™ DNA Sequencing Template Amplification Kit, available from Amersham Biosciences); TOPOTAQ™ (a hybrid polymerase that combines hyperstable DNA binding domains and the DNA unlinking activity of Methanopyrus topoisomerase, with no exonuclease activity, available from Fidelity Systems); TOPOTAQ HIFI which incorporates a proofreading domain with exonuclease activity; PHUSION™ (a Pyrococcus-like enzyme with a processivity-enhancing domain, available from New England Biolabs); any other suitable DNA polymerase, or any combination of two or more thereof.

In other embodiments, oligonucleotides and/or polynucleotides can be assembled in using other assembly methods, such as Ligase Chain Reaction (LCR; see Wiedmann et al., PCR Methods Appl. 3(4):S51-64 (1994)). More specifically, ligation-based multiplex assembly refers to a mode of multiplex assembly involving ligation of a plurality of oligonucleotides and/or polynucleotides. In some embodiments, a ligation-based assembly reaction may be used to assemble oligonucleotides that contain one or more sequence features that are known or predicted to interfere with a polymerase-based assembly reaction. Accordingly, a polynucleotide may be assembled from a plurality of intermediate fragments (e.g., fragments that are between 200 and 1,000 bases long), wherein each intermediate fragment is assembled using a polymerase-based reaction or a ligase-based reaction depending on whether the intermediate fragment contains an interfering sequence feature. In some embodiments, fragment boundaries are selected in order to isolate interfering sequences in one or a few (e.g., 2, 3, 4, or 5) fragments that are assembled using a ligation based technique. It should be appreciated that the number of fragments required to encompass all of the interfering sequence features may depend on the length of the target polynucleotide being assembled, the distribution of the interfering sequence features across the polynucleotide, and/or the length of the fragments that are being assembled by ligation. In some embodiments, the fragment sizes and boundaries are chosen in order to assemble fewer than about 50% (e.g., about 45%, 40%, 35%, 30%, 25%, 20%, 15%, 10%, 5%, or fewer) of the fragments by ligation. In some embodiments, one or more fragments assembled by ligation may be amplified in vivo in a host cell (e.g., cloned into a vector and transformed into a host cell) prior to further assembly. In certain embodiments, one or more fragments assembled by ligation may be amplified in vitro (e.g., using an amplification reaction such as a PCR or LCR reaction, etc.) prior to further assembly. For example, each of the fragments assembled by ligation and/or extension may include a tag sequence on its 5′ and/or 3′ ends, such that an oligonucleotide corresponding to the 5′ end of a ligation-assembled fragment and/or an oligonucleotide corresponding to the 3′ end of the ligation-assembled fragment can be designed to contain a segment of non-target sequence (e.g., a tag), wherein the tag sequences are identical or complementary to specific primers that that can be used as amplification primers (e.g., as PCR primers). Accordingly, the non-target sequences, or tags, can be used to amplify each ligation-assembled fragment and/or polymerase assembled fragment. In some embodiments, two or more intermediate assembled fragments (either assembled by a ligation-based or polymerase-based method) may contain common 5′ non-target sequences (e.g., a 5′ tag) and/or common 3′ non-target sequences (e.g., a 3′ tag). Accordingly, appropriate primer pairs corresponding to the common non-target sequences can be used to amplify such fragments simultaneously (e.g., in parallel or in the same reaction mixture). In some cases, non-target sequences that are common to and are used for amplification of a plurality of oligonucleotides or assembled sequences thereof (e.g., fragments of a target) may be used to amplify two or more different fragments that were assembled in different ligase-based assembly reactions. The non-target sequences subsequently may be removed from amplified polynucleotides by various methods described elsewhere herein, including, for instance, type IIS restriction enzyme, UDG, or T4 DNA polymerase based techniques. In some embodiments, one or more fragments assembled by ligation may be added to a subsequent assembly reaction (e.g., a subsequent ligation or polymerase based extension reaction) without any intervening amplification. However, it should be appreciated that fragments assembled by ligation may be concentrated and/or purified, regardless of whether they are amplified, prior to further assembly. The remainder of the fragments may be assembled by extension (e.g., in a polymerase-based assembly reaction).

In other embodiments, oligonucleotides and/or polynucleotides can be assembled in using other assembly methods, such as Iterative Capped Assembly (ICA). Iterative capped assembly can be particularly useful in the assembly of repeat-module DNA and comprises sequential ligation of monomers on a solid support together with capping oligonucleotides to increase the frequency of full-length products (see Briggs et al., Nucl. Acids Res. 40(15):e117 (2012))

In certain embodiments, assembly of the one or more double-stranded oligonucleotides comprises at least 5 cycles of denaturing, annealing and extending. For example, corresponding A and B fragment oligonucleotides were assembled with high-fidelity DNA polymerase (e.g. KAPA HIFI™) using qPCR with the corresponding A and B DNA fragments. After 5 cycles of annealing and extension, additional primers can be added, and the reaction can continue for additional cycles (typically, 20-25 cycles in addition to the first 5 cycles).

In other embodiments, assembly of the double-stranded or verified polynucleotides occurs in sets or pools of oligonucleotides. In certain embodiments, each set or pool of oligonucleotides can share a unique primer binding site that selectively amplifies that specific set or pool of oligonucleotides. The number of oligonucleotides in each set can range from approximately 100-250, approximately 250-450, approximately 450-700, approximately 700-950, approximately 950-1,200, approximately 1,200-1,450, approximately 1,450-1,675, approximately 1,675-1,800, approximately 1,800-2,025, or approximately 2,025-2,275 double-stranded or verified polynucleotides. More specifically, a set or pool can be 250, or 462, or 712, or 962, or 1,212, or 1,452, or 1,674, or 1,805, or 2,021 or 2,271 oligonucleotides. In some embodiments, assembly of the double-stranded or verified polynucleotides can occur in sets or pools of more than 2,275 oligonucleotides. In an embodiment, the method comprises assembling more than 2,000 of the double-stranded or verified polynucleotides, and wherein the double-stranded or verified polynucleotides with >50% accuracy, >60% accuracy, >70% accuracy, >80% accuracy, >90% accuracy, >95% accuracy, or >99% accuracy.

Oligonucleotide assembly or multiplex oligonucleotide assembly refers to a method wherein predetermined or predefined nucleic acid segments (i.e., the sequences of the oligonucleotides and/or polynucleotides are known and chosen before synthesis or assembly of the oligonucleotides and/or polynucleotides) can be assembled from a plurality of different starting nucleic acid segments (e.g., oligonucleotides) in a multiplex assembly reaction. Certain aspects of multiplex oligonucleotide assembly reactions are illustrated by the following description of certain embodiments of multiplex oligonucleotide assembly reactions. It should be appreciated that the description of the assembly reactions in the context of oligonucleotides is not intended to be limiting. The assembly reactions described herein may be performed using starting nucleic acids obtained from one or more different sources. As used herein, an assembly oligonucleotide has a sequence that is designed to be incorporated into the desired polynucleotide product generated during the assembly process. However, it should be appreciated that the description of the assembly reactions in the context of single-stranded oligonucleotides is not intended to be limiting. In some embodiments, one or more of the starting oligonucleotides illustrated in the figures and described herein may be provided as double stranded nucleic acids. Accordingly, it should be appreciated that where the figures and description illustrate the assembly of single-stranded nucleic acids, the presence of one or more complementary nucleic acids is contemplated. Accordingly, one or more double-stranded complementary oligonucleotides may be included in a reaction that is described herein in the context of a single-stranded assembly oligonucleotide. However, in some embodiments the presence of one or more complementary nucleic acids may interfere with an assembly reaction by competing for hybridization with one of the input assembly oligonucleotide. Accordingly, in some embodiments an assembly reaction may involve only single-stranded assembly oligonucleotide (i.e., the first plurality of single-stranded oligonucleotides may be provided in a single-stranded form without their complementary strand) as described or illustrated herein. However, in certain embodiments the presence of one or more complementary oligonucleotides may have no or little effect on the assembly reaction. In some embodiments, complementary oligonucleotide(s) may be incorporated during one or more steps of an assembly. In yet further embodiments, assembly oligonucleotide and their complementary strands may be assembled under the same assembly conditions via parallel assembly reactions in the same reaction mixture. In certain embodiments, a desired polynucleotide product resulting from the assembly of a plurality of starting oligonucleotides may be identical to the oligonucleotide product that results from the assembly of oligonucleotide that are complementary to the starting oligonucleotides (e.g., in some embodiments where the assembly steps result in the production of a double-stranded nucleic acid product). In some embodiments, an input oligonucleotide may be amplified before use. The resulting product may be double-stranded. In some embodiments, one of the strands of a double-stranded oligonucleotide may be removed before use so that only a predetermined single strand is added to an assembly reaction.

In some embodiments, the method further comprises: (c) tagging the one or more double-stranded polynucleotides, wherein the tagging comprises amplifying the one or more double-stranded oligonucleotides using a pair of tagging primers to generate one or more tagged double-stranded polynucleotides, wherein each tagging primer in the pair of tagging primers comprises: (i) a first segment comprising a unique flanking sequence, and (ii) a second segment comprising a seed sequence; (d) sequencing the one or more tagged double-stranded polynucleotides, wherein the sequencing comprises binding of the seed sequence to a sequencing platform and performing a sequencing reaction to identify one or more sequence verified polynucleotides; and (e) retrieving the one or more sequence verified polynucleotides, wherein the retrieving comprises base-pairing a complementary primer to the first segment of at least one tagging primer in the one or more sequence verified polynucleotides and, under conditions suitable and in the presence of suitable reagents, amplifying the sequence verified polynucleotides to produce one or more verified polynucleotides; or (c) phenotypic selection of functional polypeptides, wherein the phenotypic selection comprises of one or more of yeast display, phage display, mRNA display, ribosome display, mammalian cell display, bacterial cell display, emulsion-based protein selection, functional complementation of a portion of a genome, or other selection methods known to experts in the field of polypeptide evolution.

In another embodiment, the method further comprises step-wise assembly of two or more of the double-stranded or verified polynucleotides into an assembled polynucleotide product, wherein the two or more double-stranded or verified polynucleotides have overlapping regions with homology capable of annealing and at least one common primer binding site in each of the double-stranded or verified polynucleotides; and (f) combining the two more double-stranded or verified polynucleotides under conditions suitable for annealing the overlapping regions with homology and in the presence of suitable reagents for assembling an initial desired polynucleotide product by extension of the double-stranded or verified polynucleotides to produce the initial desired polynucleotide product; and (g) combining the initial desired polynucleotide product and a next double-stranded or verified polynucleotide, wherein the initial desired polynucleotide product and the next double-stranded or verified polynucleotide have overlapping regions with homology capable of annealing and at least one common primer binding site in each of the double-stranded or verified polynucleotides, and assembling the initial desired polynucleotide product and the next double-stranded or verified polynucleotide in the presence of suitable reagents for assembling the assembled polynucleotide product by extension of the initial desired polynucleotide product and the next double-stranded or verified polynucleotide; and (h) reiteratively repeating (g) to step-wise add additional next double-stranded or verified polynucleotides to the initial desired polynucleotide product to produce the assembled polynucleotide product.

In yet another embodiment, the method further comprises hierarchical assembly of two or more of the double-stranded or verified polynucleotides into an assembled polynucleotide product, wherein the two or more double-stranded or verified polynucleotides have overlapping regions with homology capable of annealing and at least one common primer binding site in each of the double-stranded or verified polynucleotides; and (f) combining the two double-stranded or verified polynucleotides under conditions suitable for annealing the overlapping regions with homology and in the presence of suitable reagents for assembling a first desired polynucleotide product by extension of the double-stranded or verified polynucleotides to produce the first desired polynucleotide product; and (g) repeating (f) with another two double-stranded or verified polynucleotides to produce a second desired polynucleotide product; (e) combining the first desired polynucleotide product and the second desired polynucleotide product, wherein the first desired polynucleotide product and the second desired polynucleotide product have overlapping regions with homology capable of annealing and at least one common primer binding site in each of the first and the second desired polynucleotide products, and assembling the first desired polynucleotide product and the second desired polynucleotide product in the presence of suitable reagents for assembling the assembled polynucleotide product by extension of the first desired polynucleotide product and the second desired polynucleotide product; and (h) repeating (f), (g) and (e) to hierarchically assemble pairs of desired polynucleotides to produce the assembled polynucleotide product.

In other embodiments, the assembled polynucleotide products are at least about 250 nucleotides, at least about 500 nucleotides, at least about 1,000 nucleotides, at least about 2,500 nucleotides, at least about 5,000 nucleotides, at least about 10,000 nucleotides, at least about 50,000 nucleotides, at least about 100,000 nucleotides or at least about 300,000 nucleotides in length. One should appreciate that there is no limit to the nucleotide length of the assembled polynucleotide products.

As used herein, step-wise assembly of two or more polynucleotides refers to the combining of two or more polynucleotides to produce a larger polynucleotide. For example, two polynucleotides (e.g. A and B) can be assembled to produce a first desired polynucleotide (e.g. AB) and a next polynucleotide (e.g. C) can be assembled to produce a next desired polynucleotide product (e.g. ABC), and then another polynucleotide (e.g. D) can be added to produce the desired polynucleotide product (e.g. ABCD). The process can be repeated as necessary to generate the desired polynucleotide product. In a further embodiment, the assembled polynucleotide products are at least about 250 nucleotides, at least about 500 nucleotides, at least about 1,000 nucleotides, at least about 2,500 nucleotides, at least about 5,000 nucleotides, at least about 10,000 nucleotides, at least about 50,000 nucleotides, at least about 100,000 nucleotides or at least about 300,000 nucleotides in length. Two, three, four, 5 or more, 10 or more, 20 or more, 50 or more, 100 or more, 250 or more, 500 or more, 1,000 or more, 1,500 or more, 2,000 or more, or 2,500 or more polynucleotides can be assembled.

As used herein, hierarchical assembly of two or more polynucleotides refers to the combining of two or more polynucleotides to produce a larger polynucleotide. For example, two polynucleotides (e.g. A and B) can be assembled to produce a first desired polynucleotide (e.g. AB) and another two polynucleotides (e.g. C and D) can be assembled to produce a second desired polynucleotide product (e.g. CD), and then the first desired polynucleotide (e.g. AB) and the second desired polynucleotide product (e.g. CD) can be assembled to produce the desired polynucleotide product (e.g. ABCD). In some embodiments, two or more subassemblies (e.g. ABCD and EFGH) can be assembled (e.g. ABDCEFGH). The process can be repeated as necessary to generate the desired polynucleotide product. In a further embodiment, the assembled polynucleotide products are at least about 250 nucleotides, at least about 500 nucleotides, at least about 1,000 nucleotides, at least about 2,500 nucleotides, at least about 5,000 nucleotides, at least about 10,000 nucleotides, at least about 50,000 nucleotides, at least about 100,000 nucleotides or at least about 300,000 nucleotides in length. Two, three, four, 5 or more, 10 or more, 20 or more, 50 or more, 100 or more, 250 or more, 500 or more, 1,000 or more, 1,500 or more, 2,000 or more, or 2,500 or more polynucleotides can be assembled. Polynucleotides and/or subassembly fragments may be combined and processed more rapidly and reproducibly to increase the throughput rate of the assembly.

In some embodiments, step-wise or hierarchical assembly can assemble 3, 4, 5, 6, 7, 8, 9, 10 or more, 15 or more, 20 or more, 25 or more, 30 or more, or 50 or more polynucleotides. For example, 15 to 20, 20 to 25, 25 to 30, 30 to 35, 35 to 40, 40 to 45, 45 to 50, or 50 or more different polynucleotides may be assembled. Each polynucleotide product being assembled may be between about 100 nucleotides long and about 1,000 nucleotides long. For example, assembled polynucleotide products can be at least about 250 nucleotides, at least about 500 nucleotides, at least about 1,000 nucleotides, at least about 2,500 nucleotides, at least about 5,000 nucleotides, at least about 10,000 nucleotides, at least about 50,000 nucleotides, at least about 100,000 nucleotides or at least about 300,000 nucleotides in length. One should appreciate that there is no limit to the nucleotide length of the assembled polynucleotide products.

In some embodiments, the tagging primer contains a unique flanking sequence or tag that can be of any suitable length that allows for generating a sufficient number of unique sequences sufficient to allow each oligonucleotide to be tagged with a unique sequence on one or both ends. For example, each of the oligonucleotides or polynucleotides can include a unique flanking sequence or tag sequence on its 5′ and/or 3′ end, such that an oligonucleotide corresponding to the 5′ end of an assembled polynucleotide product or an oligonucleotide corresponding to the 3′ end of the assembled polynucleotide product can be designed to contain a segment of non-target sequence (e.g., a tag), wherein the tag sequences are identical or complementary to specific primers that that can be used as amplification primers (e.g., as PCR primers). Accordingly, the non-target sequences, or tags, can be used to amplify each assembled polynucleotide product. In certain embodiments, each unique flanking sequence or tag has the following properties: (a) no more than 5 consecutive nucleotide residues of homoguanine or homocysteine (e.g., GGGGG or CCCCCC or GCGCGC or GGGCCC); (b) no more than 8 consecutive nucleotide residues of homoadenine or homothymine (e.g., AAAAAAAAA or ITITITITI or ATATATATA or AAAAATITT); and (c) a guanine-cysteine (GC) content between 45% and 65%. In some embodiments, the unique flanking sequence is between approximately 5 to 30, 5 to 25, 5 to 20, 5 to 15, 5 to 10, or more than 30 nucleotides in length. In other embodiments, the unique flanking sequence is approximately 5, 6, 7, 8, 9, 10, 11, 12, 13, 14, 15, 16, 17, 18, 19, 20, 21, 22, 23, 24, 25, 26, 27, 28, 29, 30 or more than 30 nucleotides in length. In an embodiment, the unique flanking sequence of the tagging primers comprise a random 13 nucleotide sequence (5′-NNNNNNNNNNNNN-3′; SEQ ID NO:26) with the following properties: (a) no more than 5 consecutive nucleotide residues of homoguanine or homocysteine; (b) no more than 8 consecutive nucleotide residues of homoadenine or homothymine; and (c) a guanine-cysteine (GC) content between 45% and 65%.

In certain embodiments, the seed sequence of the tagging primers comprise a sequence of 15-25 nucleotides capable of binding of the seed sequence to a sequencing platform and performing a sequencing reaction. For example, suitable DNA sequencing technologies that can be used in accordance with the methods described herein may include, but are not limited to, 454 pyrosequencing, Illumina Genome Analyzer, AB SOLiD, and HeliScope, nanopore sequencing methods, real-time observation of DNA synthesis, sequencing by electron microscopy, dideoxy termination and electrophoresis, microelectrophoretic methods, sequencing by hybridization, and mass spectroscopy methods. Suitable sequencing conditions can be determined by those of skill in the art based on the particular factors, and on the teachings herein.

In certain embodiments, a common primer binding site refers to a sequences used to amplify oligonucleotides that are common to all oligonucleotides of each of the separate oligonucleotides being assembled. For example, when oligonucleotide to be assembled comprises two oligonucleotides (e.g., oligonucleotide A and oligonucleotide B) a common primer binding site can refer to having the same primer binding site on all oligonucleotides for oligonucleotide A, wherein all A oligonucleotides have the same primer binding site as each other, and all oligonucleotides for oligonucleotide B should have the same primer binding site as each other. In certain embodiments, the common primer for oligonucleotide A is different than the common primer binding site for oligonucleotide B. In other embodiments, a common primer binding site refers to a sequences used to amplify oligonucleotides that are common to all oligonucleotides of each of the separate sets oligonucleotides being assembled. For example, when oligonucleotide to be assembled comprises two sets oligonucleotides (e.g., oligonucleotide set A and oligonucleotide set B) a common primer binding site can refer to having the same primer binding site on all oligonucleotides for oligonucleotide set A, wherein all set A oligonucleotides have the same primer binding site as each other, and all oligonucleotides for oligonucleotide set B should have the same primer binding site as each other. In certain embodiments, the common primer for oligonucleotide set A is different than the common primer binding site for oligonucleotide set B.

After verifying the accuracy of each of the polynucleotides by sequencing the set of polynucleotides or assembled polynucleotide products, the sequence of each member of a polynucleotide set or assembled polynucleotide products are known, and the desired, accurate sequence or sequences are identified and selected for recovery and amplification. Methods for selection, recovery and amplification of one or more desired polynucleotide or assembled polynucleotide product include any suitable selection method to exploit the unique flanking sequence to selectively target the desired polynucleotide or assembled polynucleotide product, which are confirmed as accurate the sequence or sequences. Such selection methods are referred to herein as “dial-out retrieval” (see U.S. Patent Application Publication No. 20120283110, which is incorporated by reference). Suitable dial-out selection methods may include, but are not limited to, hybridization-based capture methods, 2-primer based PCR methods directed to members of nucleic acid libraries that are tagged with two sets of adaptor sequences that include two dial-out tag sequences, 1-primer PCR methods directed to members of nucleic acid libraries that are tagged with one set of adaptor sequences having a single dial-out tag sequence, linear amplification, multiple displacement amplification, rolling circle amplification, and ligation-based methods (e.g., selective circularization methods, molecular inversion probes).

In an embodiment, the retrieval method used for selection, recovery and amplification of one or more desired polynucleotide or assembled polynucleotide product may be a method of selective amplification referred to herein as “dial-out PCR.” A dial-out PCR method is a clone-free and highly parallel method for obtaining sequence-verified nucleic acids (e.g., oligonucleotides or desired polynucleotide or assembled polynucleotide products). Any suitable PCR protocol known in the art may be used to amplify the sequence-verified target desired polynucleotide or assembled polynucleotide product acid including, but not limited to those methods described in the Examples below.

In other embodiments, the retrieval method used for selection, recovery and amplification of one or more desired polynucleotide or assembled polynucleotide product may be a method of phenotypic selection of functional polypeptides, wherein the phenotypic selection comprises of one or more of yeast display (see Tinberg et al., Nature 501(7466):212-16 (2013)), phage display, mRNA display, ribosome display, mammalian cell display, bacterial cell display, emulsion-based protein selection, functional complementation of a portion of a genome, or other selection methods known to experts in the field of polypeptide evolution. For example, variants of an essential polynucleotide sequence from the E. coli genome could be assembled into a polynucleotide product and then introduced into the E. coli genome while deleting the endogenous E. coli polynucleotide sequence. In such an example, the surviving cells would have the desired polynucleotide product sequence that could functionally replace the original. In another embodiment, a high-throughput screen for protein function could be performed.

In certain embodiments, in order to obtain a desired polynucleotide product from a multiplex oligonucleotide assembly, a purification step may be used to remove starting oligonucleotides and/or incompletely assembled fragments. In some embodiments, a purification step may involve chromatography, electrophoresis, or other physical size separation technique (e.g., AMPure®, XP beads; Agencourt). In certain embodiments, a purification step may involve amplifying the full length product. For example, a pair of amplification primers (e.g., PCR primers) that correspond to the predetermined 5′ and 3′ ends of the polynucleotide product being assembled will preferentially amplify full length product in an exponential fashion. It should be appreciated that smaller assembled products may be amplified if they contain the predetermined 5′ and 3′ ends. However, such smaller-than-expected products containing the predetermined 5′ and 3′ ends should only be generated if an error occurred during assembly (e.g., resulting in the deletion or omission of one or more regions of the target nucleic acid) and may be removed by size fractionation of the amplified product. Accordingly, a preparation containing a relatively high amount of full length product may be obtained directly by amplifying the product of an assembly reaction using primers that correspond to the predetermined 5′ and 3′ ends. In some embodiments, additional purification (e.g., size selection) techniques may be used to obtain a more purified preparation of amplified full-length nucleic acid fragment.

One of skill in art would appreciate that the polynucleotide products generated by the methods described herein may be useful for a range of applications involving the production and/or use of synthetic nucleic acids. As described herein, these methods provide for assembling synthetic nucleic acids with increased efficiency, with significantly greater accuracy and with significant less costs. The resulting polynucleotide products may be further amplified in vitro (e.g., using PCR), or in vivo (e.g., via cloning into a suitable vector), and isolated and/or purified. An assembled polynucleotide product may be transformed into a host cell (e.g., a prokaryotic, eukaryotic, or other host cell). Accordingly, the polynucleotide products may be used to produce recombinant organisms. In some embodiments, a polynucleotide products may be an entire genome or large fragments of a genome that are used to replace all or part of the genome of a host organism. Recombinant organisms also may be used for a variety of research, industrial, agricultural, and/or medical applications. In other embodiments, antibodies can be made against polypeptides or fragment(s) thereof encoded by one or more of the polynucleotide products. In certain embodiments, the polynucleotide products may be provided as libraries for screening in research and development (e.g., to identify potential therapeutic proteins or polypeptides, to identify potential protein targets for drug development). In some embodiments, the polynucleotide products may be used as a therapeutic (e.g., for gene therapy, or for gene regulation).

### Exemplary Aspects

Below are examples of specific aspects for carrying out the present invention. The examples are offered for illustrative purposes only, and are not intended to limit the scope of the present invention in any way. Efforts have been made to ensure accuracy with respect to numbers used (e.g., amounts, temperatures, and the like), but some experimental error and deviation should, of course, be allowed for.

### Materials and Methods

### Target Designs

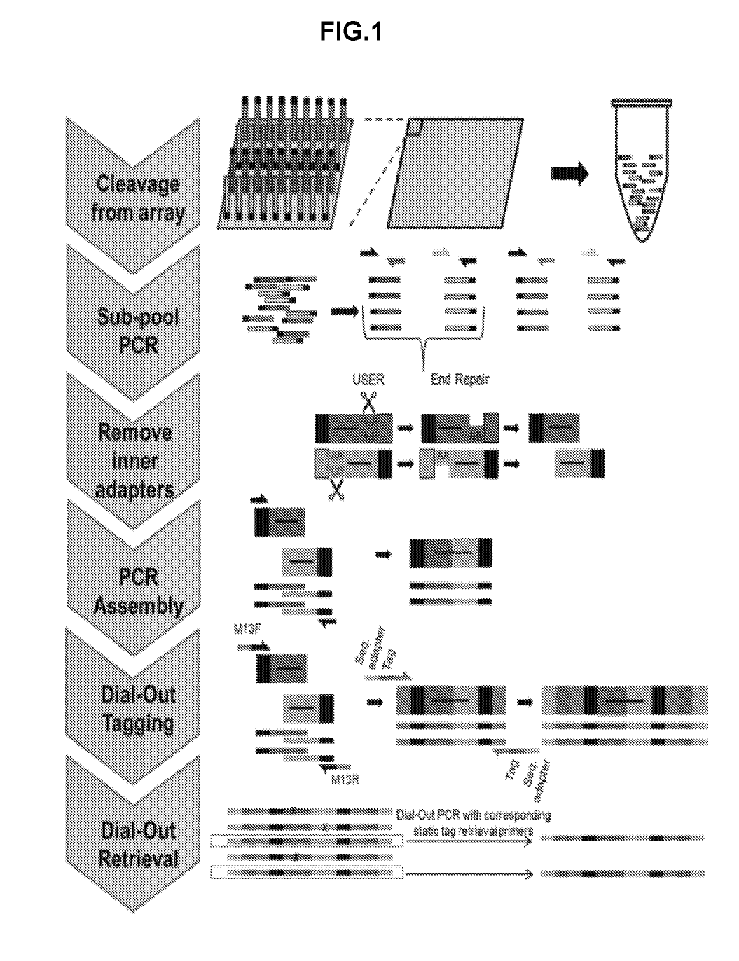

Target polynucleotide sequences ranged from 156-216 bases of unique sequence and were split into 10 sets. Each oligonucleotide target was fragmented into two pieces (A and B) using a custom python script that determines overlaps with the least chance of cross-hybridization (see Klein et al., Nucleic Acids Research, 44(5):e43, Supplementary materials). Briefly, the following procedure was automated using python: bases for the overlap region were dynamically added starting from the midpoint-7 position until the melting temperature was >56° C. The overlap fragment was then checked against all sequences in the set and accepted if <15 consecutive bases aligned to any other sequence. To quickly evaluate alignments against all sequences in a given set, a simple sliding algorithm was utilized, which scores the longest consecutive alignments. If the overlap sequence failed these conditions, then up to 6 codons were swapped out at random within this sequence region, and if the melting temperature was still >56° C., the alignment step was repeated. If conditions still were not met, then the starting position for the overlap region was shifted and the procedure was repeated. A window of 6 bases around the starting position was explored. A common 18 bp adapter was appended to the 5′ end of A fragments and 3′ end of B fragments. Two adenines were appended to the 3′ end of A fragments, and two thymines were appended to the 5′ end of B fragments. Finally, depending on length, either one or two pool-specific primers site(s) were added to all oligonucleotide designs, and random bases were added on the 3′ side to reach 160 bases for each oligonucleotide design (see FIG. 1). The pools of oligonucleotides were then synthesized by CustomArray in duplicate to decrease oligonucleotide dropout and increase uniformity.

### Pairwise Oligonucleotide Assembly

Targets were separated into sets of complexity ranging from 131-250. Each pool of A and B fragments was amplified off of the array using one common primer and one pool-specific uracil-containing primer with the KAPA HIFI™ Hot-Start Uracil+ Readymix. Quantitative PCR (qPCR) was performed in 25 μL reactions with SYBR™ Green on a MiniOpticon Real-Time PCR system (Bio-Rad) with 2.5 ng template. Each pool was pulled from the thermocycler one cycle before plateauing, purified with 1.8× AMPure® XP beads and eluted in 20 μL. Two microliters of NEB USER™ enzyme was mixed with the purified PCR pools, and incubated at 37° C. for 15 minutes, followed by 15 minutes at room temperature. The pools were then treated with NEBNext® End Repair Module per manufacturer's protocol to remove adapter sequences. The pools were purified and concentrated in 10 μL using Zymo DNA Clean and Concentrator™.

Corresponding A and B fragment libraries were assembled with KAPA HIFI™ Hotstart Readymix (KAPA Biosystems) using qPCR with a total of 1.5 ng of the purified, corresponding input DNA pools. After 5 cycles of annealing and extension, 7.5×10⁻¹² moles of each outer primer (YF-pu1L and YR-pu1R) were added, and the reaction was continued for additional cycles. Reactions were monitored on a real-time qPCR instrument, and terminated one or several cycles before plateauing. Typically, this required 20-25 cycles in addition to the first 5 cycles. For both phases, the following protocol was used: (i) 95° C. for 2 minutes, (ii) 98° C. for 20 seconds, (iii) 65° C. for 15 seconds, (iv) 72° C. for 45 seconds, (v) repeat steps ii-iv. Reactions were then purified with 1.8× AMPure® XP beads and eluted in 20 μL.

Two nanograms of the purified reaction were used in another real-time PCR with KAPA HIFI™ Hotstart Readymix with Pu1L Flowcell and Pu1R Flowcell primers. Reactions were pulled from the cycler one cycle before plateauing, purified with 1.8× AMPure® XP beads, and sequenced on an Illumina MiSeq with paired end 155 bp reads with Pu1 Sequencing F, Pu1 Sequencing R and Pu1 Sequencing I (Table 1). For complex sets of up to 2,271 targets, input DNA from the corresponding sub pools were mixed together, maintaining the same total amount of 1.5 ng input DNA.

TABLE 1
Primers used in methods

| Primer | Sequence |
|---|---|
| (SEQ ID NO.) | (5′ to 3′) |
| Uracil-containing A | GCGAN13UU |
| fragment | |
| (SEQ ID NO: 01) | |
| Uracil-containing B | CCATN13UU |
| fragment | |
| (SEQ ID NO: 02) | |
| A fragment unique F | CCATN13 |
| (SEQ ID NO: 03) | |
| B fragment unique R | GCGAN13 |
| (SEQ ID NO: 04) | |
| YF | GTTTTCCCAGTCACGAC |
| (SEQ ID NO: 05) | |
| YR | CAGGAAACAGCTATGAC |
| (SEQ ID NO: 06) | |
| Dial-Out_Tags_F | CGACAGTAACTACACGGCGAN13GTTTTCCCAGTCACGAC |
| (SEQ ID NO: 07) | |
| Dial-Out_Tags_R | GTAGCAATTGGCAGGTCCATN13CAGGAAACAGCTATGAC |
| (SEQ ID NO: 08) | |
| Dial-Out_Flow_Cell_F | AATGATACGGCGACCACCGAGATCTACACACGTAGGCCGA |
| (SEQ ID NO: 09) | CAGTAACTACACGGCGA |
| Dial-Out_Flow_Cell_R | CAAGCAGAAGACGGCATACGAGATNNNNNNNNNGACCGTC |
| (SEQ ID NO: 10) | GGCGTAGCAATTGGCAGGTCCAT |
| Dial-Out_Sequencing_F | ACGTAGGCCGACAGTAACTACACGGCGA |
| (SEQ ID NO: 11) | |
| Dial-Out_Sequencing_R | GACCGTCGGCGTAGCAATTGGCAGGTCCAT |
| (SEQ ID NO: 12) | |
| Dial-Out_Sequencing_I | ATGGACCTGCCAATTGCTACGCCGACGGTC |
| (SEQ ID NO: 13) | |
| YF-pu1L | CTAAATGGCTGTGAGAGAGCTCAGGTTTTCCCAGTCACGA |
| (SEQ ID NO: 14) | C |
| YF-pu1R | ACTTTATCAATCTCGCTCCAAACCCAGGAAACAGCTATGA |
| (SEQ ID NO: 15) | C |
| Pu1L_Flow_Cell | AATGATACGGCGACCACCGAGATCTACACACGTAGGCCTA |
| (SEQ ID NO: 16) | AATGGCTGTGAGAGAGCTCAG |
| Pu1R_Flow_Cell | CAAGCAGAAGACGGCATACGAGATNNNNNNNNNGACCGTC |
| (SEQ ID NO: 17) | GGCACTTTATCAATCTCGCTCCAAACC |
| Pu1_Sequencing_F | ACGTAGGCCTAAATGGCTGTGAGAGAGCTCAG |
| (SEQ ID NO: 18) | |
| Pu1_Sequencing_R | GACCGTCGGCACTTTATCAATCTCGCTCCAAACC |
| (SEQ ID NO: 19) | |
| Pu1_Sequencing_I | GGTTTGGAGCGAGATTGATAAAGTGCCGACGGTC |
| (SEQ ID NO: 20) | |

The uracil-containing primers and Dial-Out tags both include in-silico designed 13-mer barcodes, represented as N13 in this table. These were used for amplifying sub-pools from the array, as well as for tagging assembled constructs for Dial-Out PCR.

### In Silico Design of Static Tag Library

Random 13-mer sequences were generated and screened them for several properties: no homoguanine or homocytosine stretches >5 bp, no homoadenine or homothymine stretches >8 bp and GC content between 45% and 65%. The 13-mers passing this filter were added to a potential set if the last 10 bases had <90% nucleotide identity with any other forward, reverse, complement or reverse complement already in the list. This pipeline was repeated several times, ultimately with 1.2 million iterations, to generate a library of 7,411 13-mers.

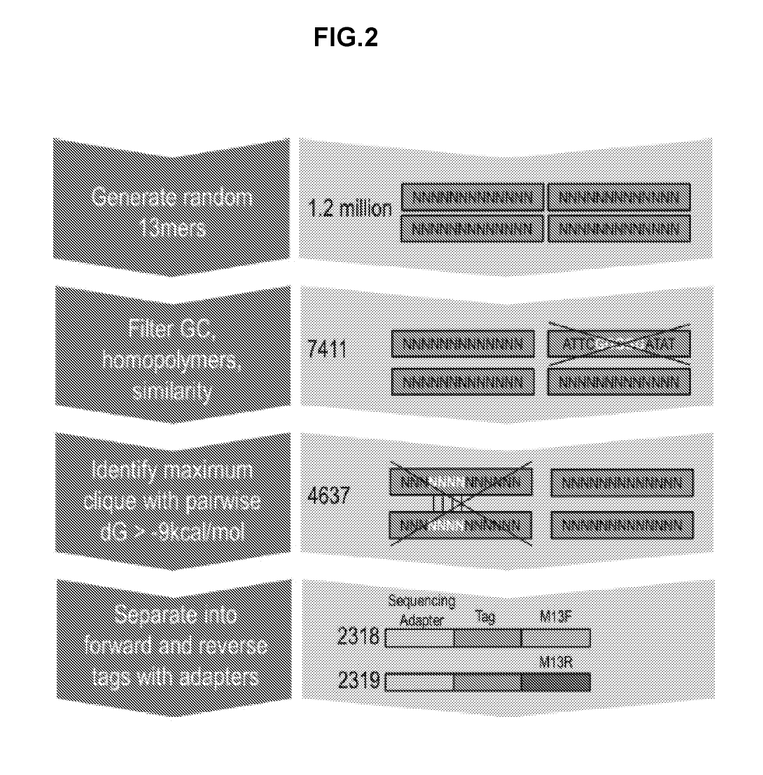

The Gibbs free energy of every possible primer pair was calculated using Unafold with the following settings: —NA=DNA, —run-type=html, —Ct=0.000001, —sodium=0.050, —magnesium=0.002. All 13-mer pairs with dG>-9 kcal/mol were indexed and added to a Matrix Market Matrix. The maximum library of 13-mers with all pairwise dG>-9 kcal/mol was then identified using the Parallel Maximum Clique Library (arXiv: 1302.6256). The indexed 13-mers were converted back to their corresponding sequences, and an additional step was applied to remove any primers with potential homodimers. This left a set of 4,637 13-mers, which was split into a forward library of 2,318 tags and a reverse library of 2,319 tags, with a total tag complexity of 5,444,982 (FIG. 2).

To the forward 13-mers, 5′-CGACAGTAACTACACGGCGA-3′ (SEQ ID NO:21) was added to the 5′ end as a bridge for the flow cell adapter, and M13F (5′-GTITCCCAGTCACGAC-3′; SEQ ID NO:22) was added to the 3′ end as the Dial-Out seed sequence. To the reverse 13-mers, 5′-GTAGCAATFGGCAGGTCCAT-3′ (SEQ ID NO:23) was used as the bridge and M13R (5′-CAGGAAACAGCTATGAC-3′; SEQ ID NO:24) was used as the seed sequence.

### Design and Synthesis of Dial-Out Retrieval Primers

To the forward For each 13-mer, the Tm was calculated using Tm=81.5+16.6×log 10[Na+]+41×(GC)−600 n. Primer sequences were determined by recursively adding 2 bp from the bridge sequence to the 5′ end of the primer until the Tm was between 58° C. and 61° C. After this procedure, all primers were 17 nucleotides or 19 nucleotides long, with Tm between 58.2° C. and 60.6° C. Primers were ordered from Integrated DNA Technologies (IDT) in 96-well plate format with standard desalting.

### Static Tag Library Synthesis and Preparation

The 4,637 tags were synthesized using CustomArray's semiconductor electrochemical process in duplicate. Forward and reverse tag sets were amplified in 24 parallel 50 μL reactions from 1.25×10⁻¹⁴ moles template/reaction using FP: 5′-CGACAGTAACTACACGGCGA-3′ (SEQ ID NO:21) and RP: 5′-GTCGTGACTGGGAAAAC-3′ (SEQ ID NO:25) with KAPA HIFI™ Hotstart Readymix for 17 cycles. Ten nanomolar PCR products were digested with NEB lambda exonuclease following manufacturer's protocol. A 113 ng sample was mixed with equivolume Novex® TBE Urea Sample Buffer and heated at 70° C. for 3 minutes, then chilled on ice. Samples and ladder were run on a Novex® TBE Urea Gel, and the corresponding 50 bp band was cut. The bands were diced and spun through a 600 ml Eppendorf with a hole from a 22 gauge needle. The slurries were incubated with TE buffer at 65° C. for 2 hours and purified on a Spin-X column (Corning). Purified DNA was treated with the Qiagen nucleotide removal kit per manufacturer's protocol.

### Tagging of Assembled Targets

Several concentrations of tags and input were tested for optimal tagging with several different polymerases (Table 2). It was identified that 8.5×10⁻¹⁴ moles of tags with 3 ng input (a 10:1 tag:input molecular ratio) with KAPA HIFI™ HotStart Readymix, yielded optimal performance. During the assembly process, targets were amplified with primers containing M13F and M13R, following the assembly protocol above. Libraries were purified with 1.8× AMPure® XP beads and eluted in 20 μL. Three nanograrns of purified assembly library was tagged with 8.5×10⁻¹⁴ moles of dial-out tags (Dial-Out Tags F and Dial-Out Tags R) using KAPA HIFI™ HotStart Readymix using qPCR and the following cycling conditions: (i) 95° C. for 2 minutes, (ii) 98° C. for 20 seconds, (iii) 65° C. for 15 seconds, (iv) 72° C. for 45 seconds, (v) repeat steps ii-iv 30 times and (vi) 72° C. for 5 minutes and (vi) 72° C. for 5 minutes. After the first 5 cycles, the reaction was paused, and 1.5×10⁻¹¹ moles of barcoded forward and reverse flow-cell primers (Dial-Out Flow Cell F and Dial-Out Flow Cell R) were added. The tagged libraries were removed from the cycler one cycle before plateauing, and purified using 1.8× AMPure® XP beads.

TABLE 2
Optimizing polymerase and tag concentration.

| | | % Yield | % of molecules |
|---|---|---|---|
| | | (Targets with | with unique dial- |
| | Tags in | perfect | out tag |
| Polymerase | moles | assemblies) | combination |
| KAPA HIFI ™ | 8.5E−14 | 91.3 | 81.5 |
| KAPA HIFI ™ | 4.25E−13 | 91.3 | 85.7 |
| KAPA HIFI ™ | 8.5E−13 | 84.6 | 89.3 |
| KAPA HIFI ™ | 1.0E−12 | 90.3 | — |
| KAPA2G ™ Robust | 8.5E−14 | 64.4 | — |
| KAPA2G ™ Multiplex | 8.5E−14 | 85.5 | — |

The effect of several different tag concentrations on assembly yield with KAPA HIFI™ polymerase was first tested. For this dataset, the M13 sequences were present on the oligonucleotides, and tags were introduced during assembly. The greatest yield was obtained with approximately a 10:1 molar ratio of tag:template, without a large loss in the percentage of unique tag pairs. This ratio was next tested with two different polymerases, KAPA2G™ Robust and KAPA2G™ Multiplex.

### Sequence-Verification of Dial-Out Tagged Targets

The tagged library was sequenced on an Illumina MiSeq with PE 155 bp reads using Dial-Out Sequencing F, Dial-Out Sequencing R and Dial-Out Sequencing I primers. Reads were merged with PEAR using default settings and tag pairs for all reads were identified. Using a custom python script (see Klein et al., Nucleic Acids Research, 44(5):e43, Supplementary materials), all reads containing sequence-verified constructs were identified, and their corresponding tag pairs. One correctly-assembled molecule per target meeting the following criteria was randomly selected for retrieval: (i) containing a unique tag set not identified on any other molecule and (ii) represented in at least 5 sequencing reads.

### Dial-Out Retrieval

Selected oligonucleotides were retrieved via PCR with KAPA HIFI™ Hotstart Readymix using real-time PCR with 0.135 ng template and 1.5×10⁻¹¹ moles each of the corresponding forward and reverse dial-out retrieval primer with the following conditions: (i) 95° C. for 3 minutes, (ii) 98° C. for 20 seconds, (iii) 65° C. for 15 seconds, (iv) 72° C. for 40 seconds, (v) repeat steps ii-iv 34 times and (vi) 72° C. for 5 minutes. Reactions were removed from the cycler just before plateauing, purified with 1.8× AMPpure® and quantified using a Qubit™ (Invitrogen). Equal concentrations of each retrieval reaction were mixed for sequencing.

### Analysis of Average Nucleotide Accuracy

All sequencing reads were aligned to a reference of intended target sequences using BWA v.0.7.3. The average nucleotide accuracy was calculated from bases with aligned reads with base and quality mapping score >20. To compare accuracy rates between experiments, error rates were analyzed for set 5 before and after assembly. Exact Poisson Tests were performed on the 15,935,028 bases of the assembled set and 9,325,493 bases of the corresponding oligonucleotide pools passing our quality cutoffs, and on the 1,546,665 bases of the overlapping region in the assembled set and 1,617,760 in the oligonucleotide pools.

### Example 1. Assembling Targets in Sets of 131-250

2,271 targets ranging from 192-252 bases were derived (156-216 of unique sequence) to assemble from array derived oligonucleotides. All targets consisted of a unique sequence flanked by the same 18 bp 5′ and 3′ common adapters. Each target sequence was split into two fragments, A and B, containing an overlap region with a Tm >56° C. The 2,271 target sequences were split into 10 sets of 131-250 targets, and each set received unique adapters flanking the 3′ end of the A fragments and the 5′ end of the B fragments designed for uracil incorporation (FIG. 1). The corresponding oligonucleotides (160-mers with buffer sequence) were synthesized by CustomArray in duplicate to reduce oligonucleotide dropout and increase uniformity.

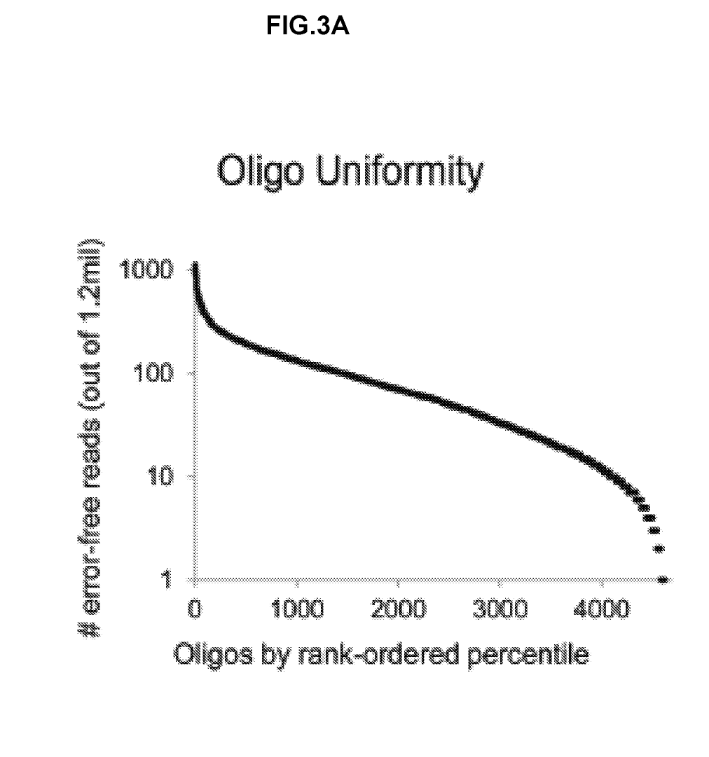

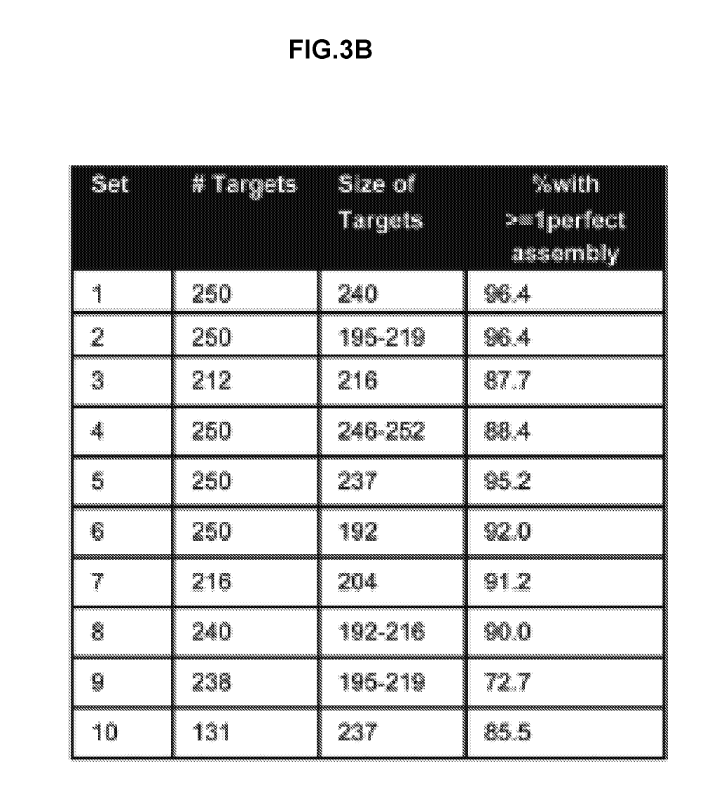

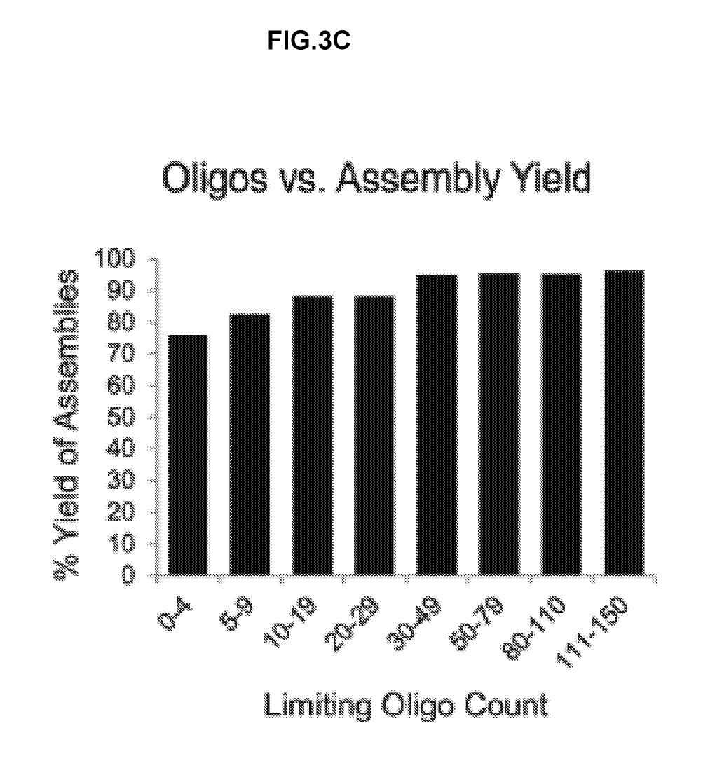

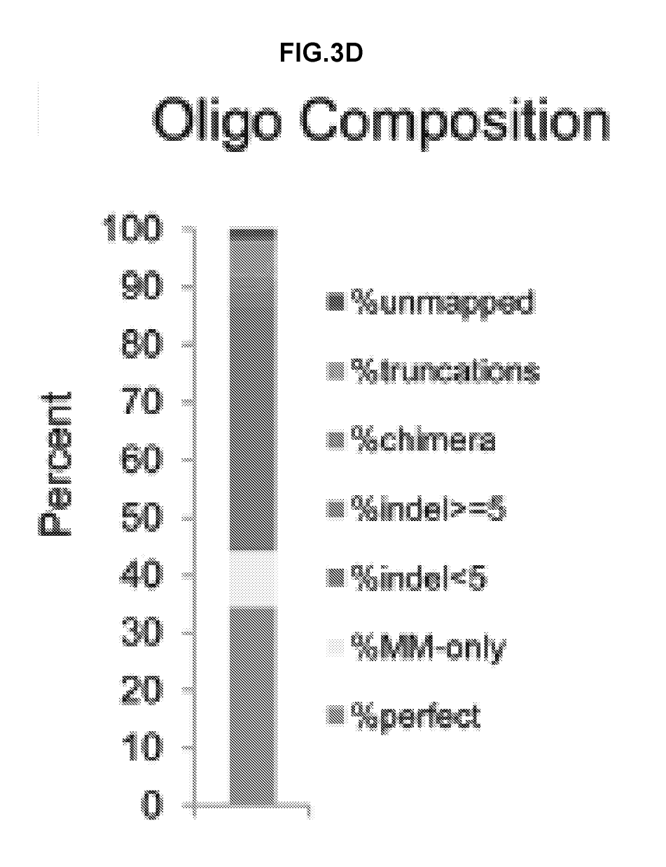

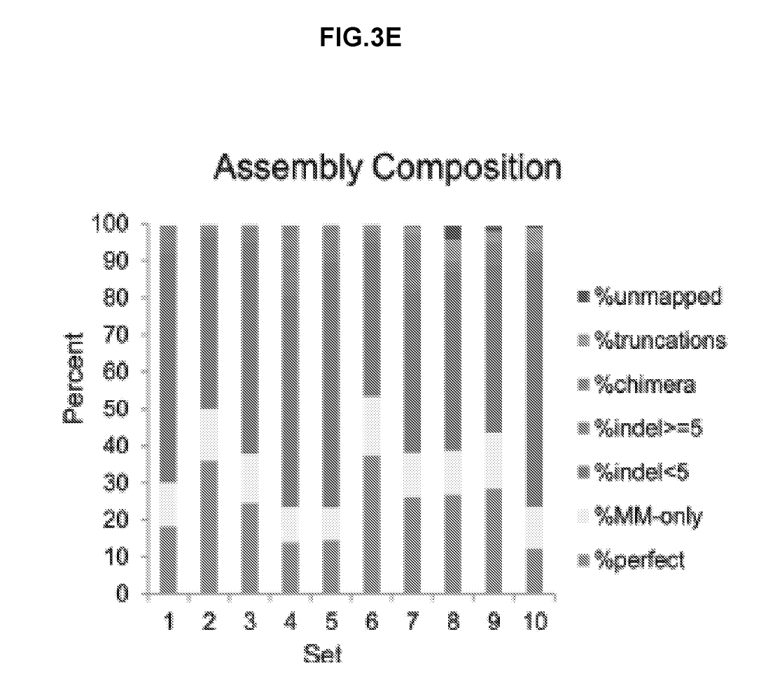

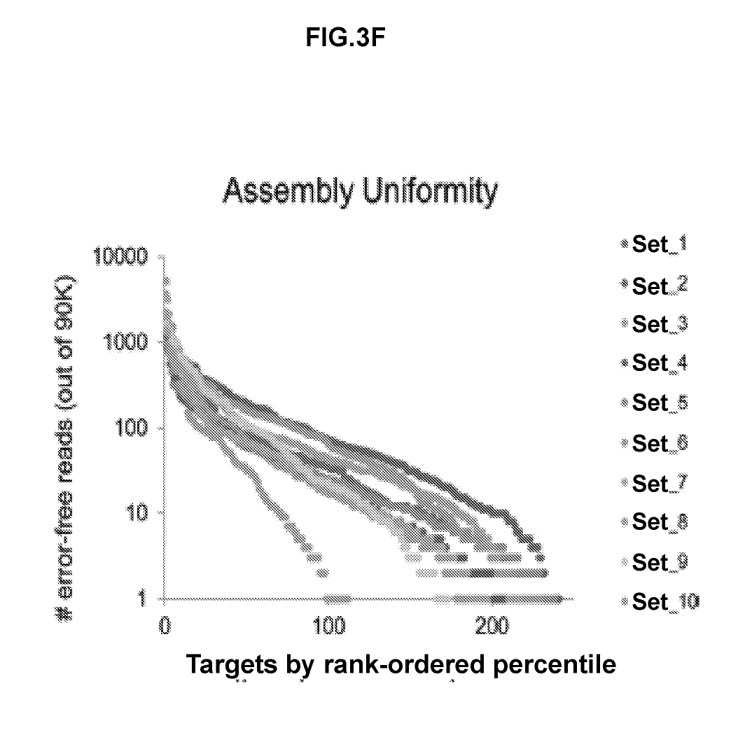

Each pool of oligonucleotides was first amplified off the array with a sub pool specific primer (A fragment unique Forward or B fragment unique Reverse) on one end and a common primer (YF/YR) on the other (Table 1). Sequencing of the oligonucleotide library showed good uniformity, with an interquartile range of 5.5 (FIG. 3A).

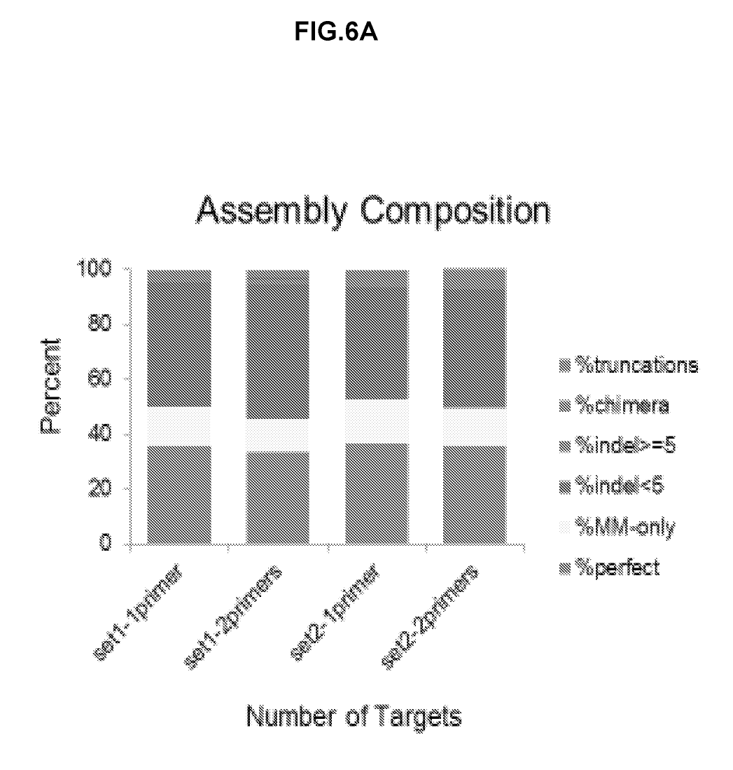

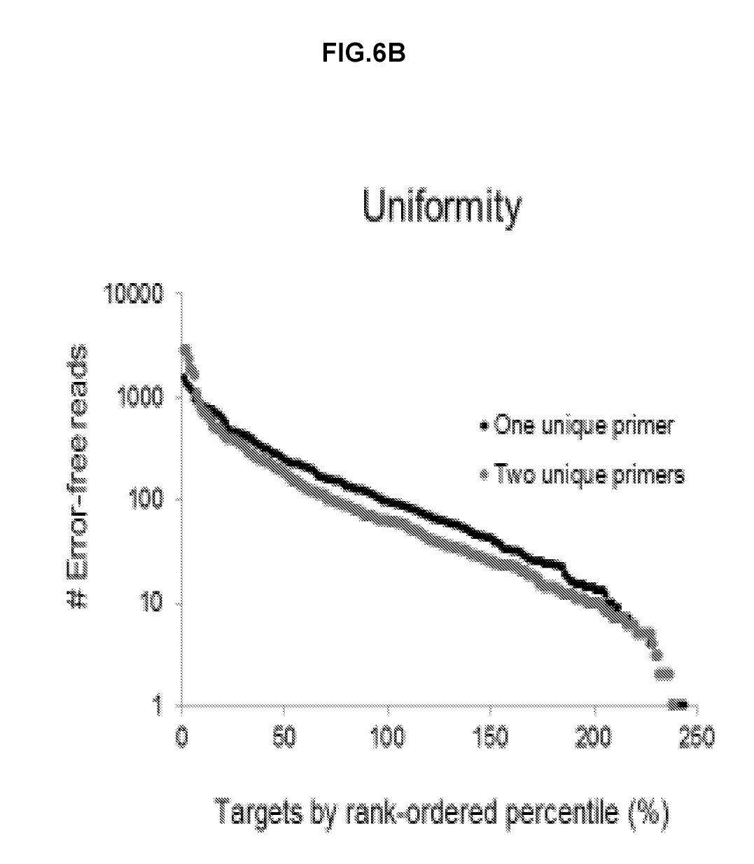

The oligonucleotide pools provided by CustomArray were then amplified using either Uracil-containing A fragment primer and common primer YF or Uracil-containing B fragment primer and common primer YR (Table 1), and the corresponding specific adapters were removed with Uracil Specific Excision Reagent (USER™). For two pools, amplifying oligonucleotides were tested with either one or two unique primer sites and observed no difference in assembly composition or uniformity (FIG. 6A and FIG. 6B). The corresponding A and B fragments were mixed for each set of targets and assembled through 5 cycles of annealing with extension and approximately 25 cycles of amplification with KAPA HIFI™. In all cases, the correct size band was observed. Each assembled set was barcoded and sequenced.

For each set, error-free assembled constructs for 72.7-96.4% of targets were identified at a sequencing depth of 90,000 reads (FIG. 3B). For each target, the number of error-free reads was examined for the corresponding A and B oligonucleotides (out of 1.2 million reads). Of the 223 targets with no error-free assemblies identified, 55 (24.7%) fell in the bottom 10th percentile of limiting oligonucleotide concentration (<6 error-free reads out of 1.2 million) and 97 (43.5%) fell in the bottom 20th percentile of limiting oligonucleotide counts (<11 error-free reads out of 1.2 million). FIG. 3C shows higher yield (% of targets with at least one perfect assembled sequence) for targets assembled from better-represented limiting oligonucleotides in the array pool, suggesting that increasing oligonucleotide uniformity would likely improve the yield of full-length designs. The composition of the raw oligonucleotide pools and the assembled target libraries (FIG. 3D and FIG. 3E) was examined next. A total of 23.8% of molecules represented error-free assemblies, 36.2% contained indel-free assemblies and 53.4% contained small indels (<5 bp). An additional 2.3% contained large indels (>5 bp), 4.8% contained chimeras, 2.1% contained truncated constructs and 0.6% unmapped reads. Within each set, 6 of 10 sets had <15-fold difference in the interquartile range. While this may be an issue for some applications, the uniformity is tight enough to use the sets directly for some downstream screening applications, such as functional protein screens. Uniformity plots are shown in FIG. 3F.

Of the 2,271 targets synthesized in individual sets, error-free constructs were assembled for 2,055 (90.5%). Much of the drop-out appears to be due to poor representation of the corresponding oligonucleotides in the array pool (FIG. 3C). Additionally, the majority of errors identified in the assembled sets are likely from the array-synthesis, since similar error profiles are identified in the oligonucleotide pools (FIG. 3D and FIG. 3E). Chimeric assembly (assembly of the wrong A and B fragments) is rare.

### Example 2. Multiplex Assembly of 2,271 Pairs of Fragments

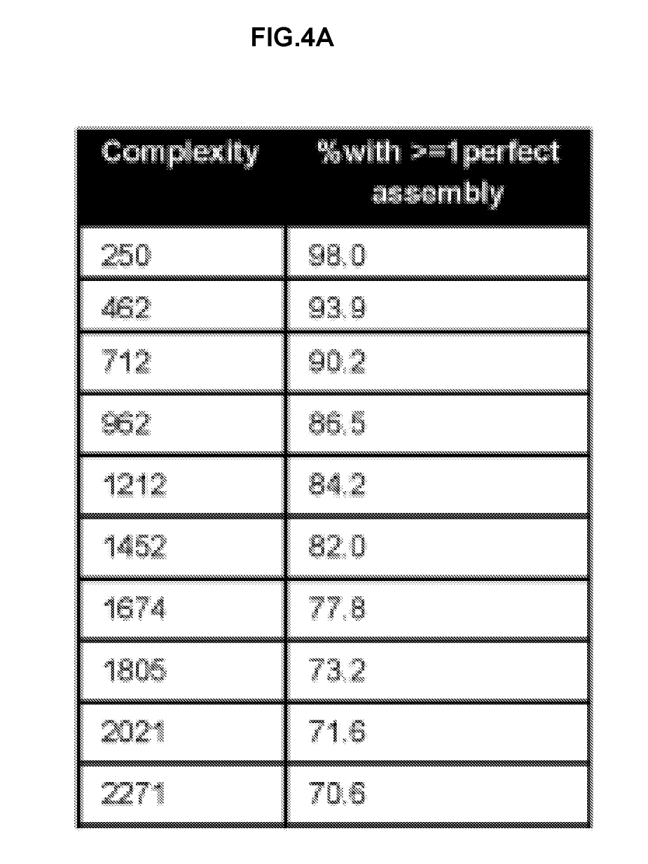

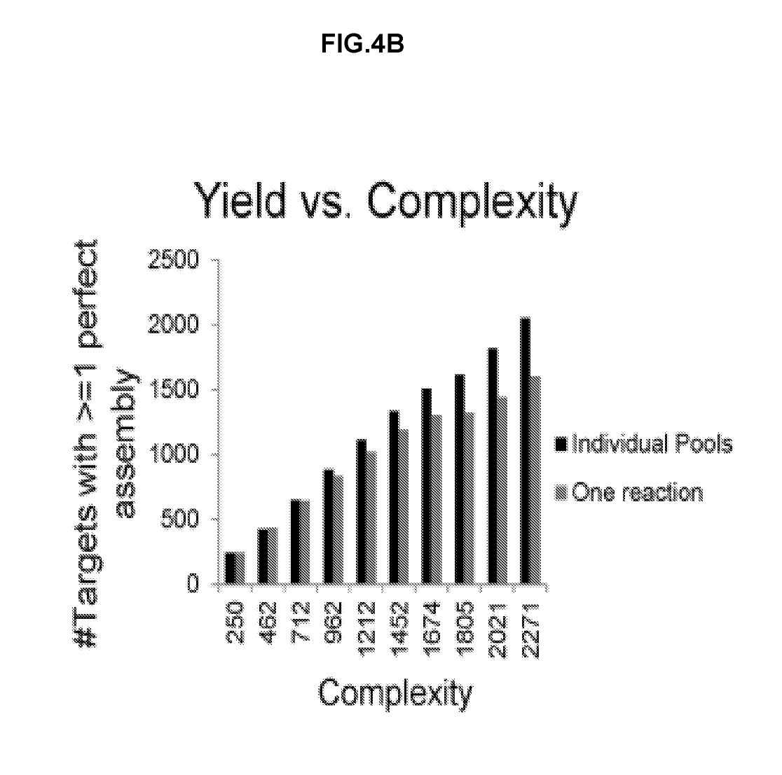

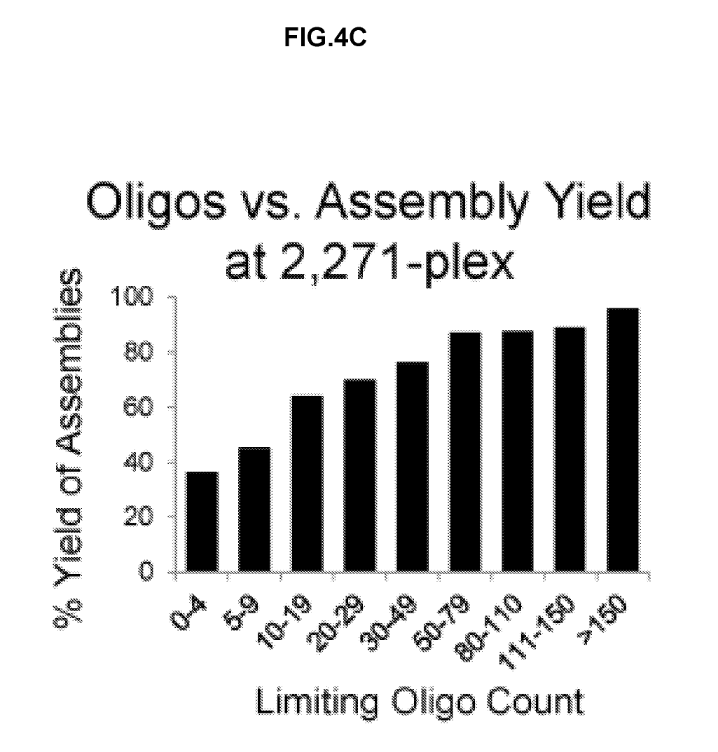

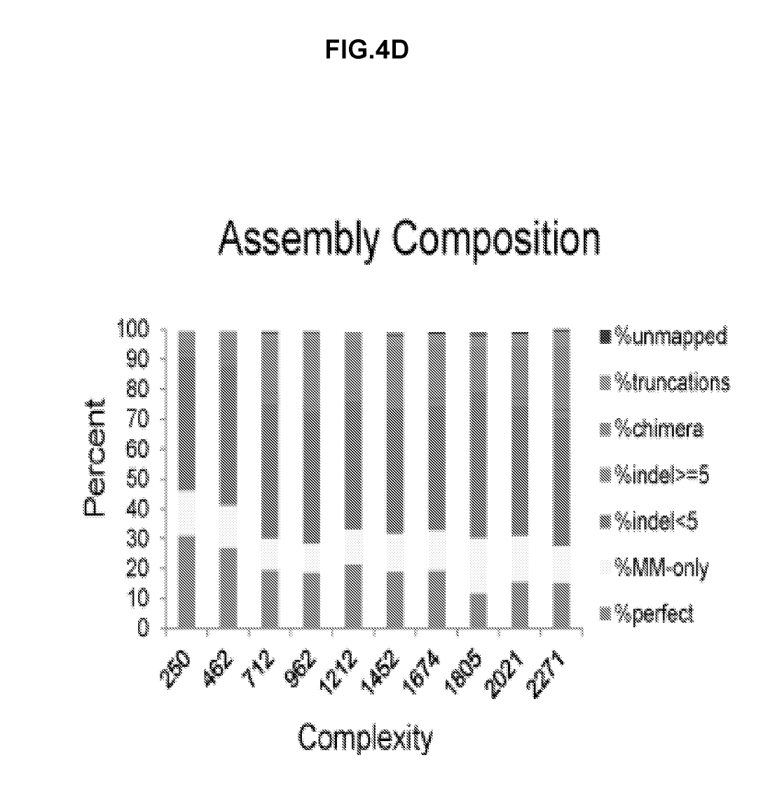

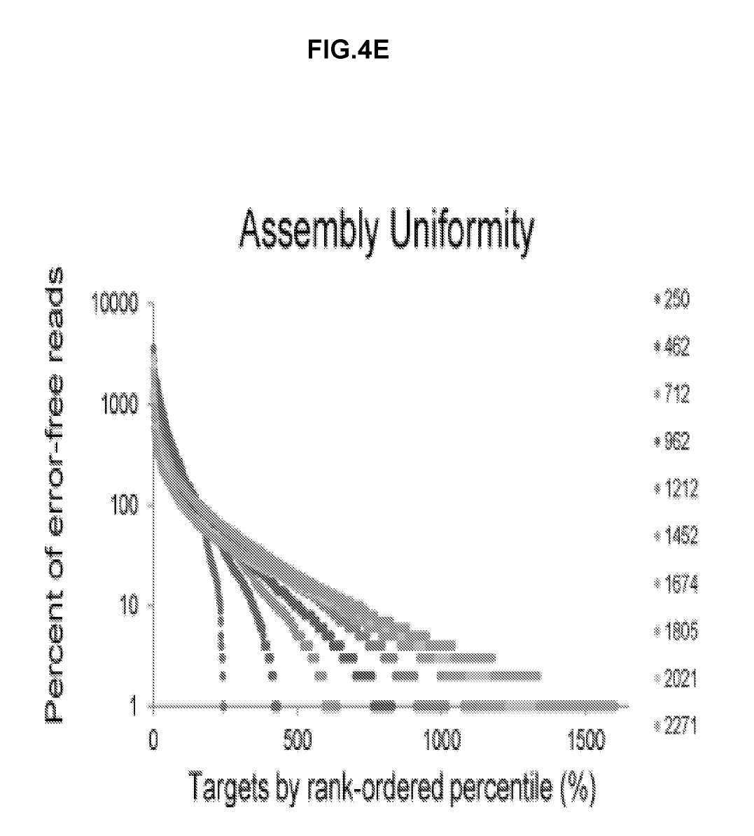

To test the limitations of the assembly protocol, complexity was increased by adding one additional set at a time, up to a complexity of 2,271 designs. At a complexity of 2,271, error-free constructs were assembled for 70.6% of targets at a sequencing depth of 300,000 reads (FIG. 4A and FIG. B). An even greater correlation was observed between yield and representation of the limiting oligo in the array pool compared to the smaller sets (FIG. 4C).

It is possible that increasing complexity could affect the composition of assembled libraries. While the two lowest complexity sets (250 and 462 targets) show the highest percentage of perfect and indel-free reads, it is likely due to the fact that these two sets are composed of sets 2 and 3, which individually showed high percentages of perfect and indel-free reads (FIG. 3E). The remaining libraries all share similar compositions. For all complexity levels, 11.8-31.3% of reads represented perfect constructs, 10.0-18.7% represented constructs with mismatches only, 41.4-48.5% represented small indels, 2.6-3.5% represented large indels, 3.7-21.5% represented chimeras, 2.5-4.9% represented truncations and 0.1-0.7% unmapped reads (FIG. 4D). Within each set, there was a 10- to 34-fold difference in the interquartile range (uniformity plots are shown in FIG. 4E).

### Example 3. Error Correction of Assemble Targets

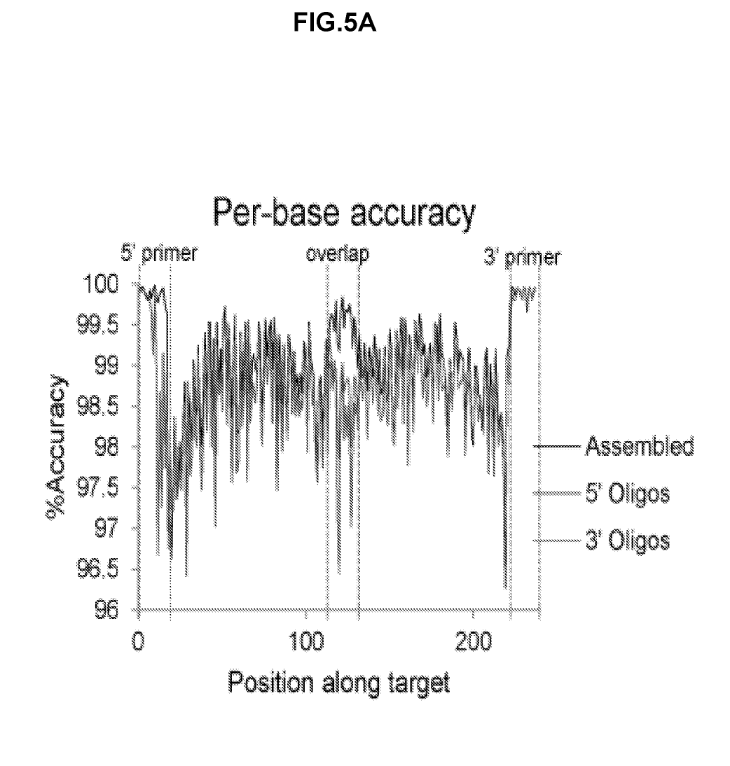

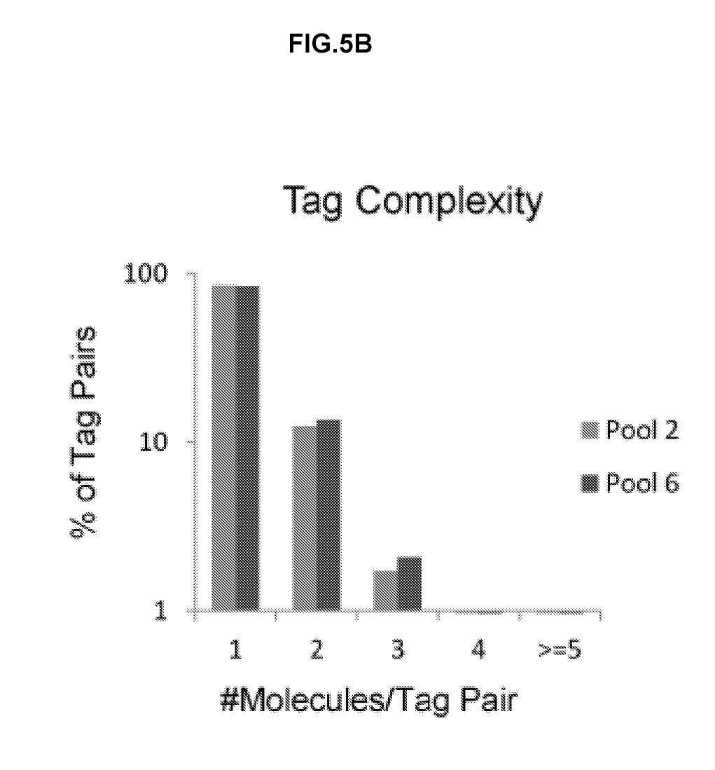

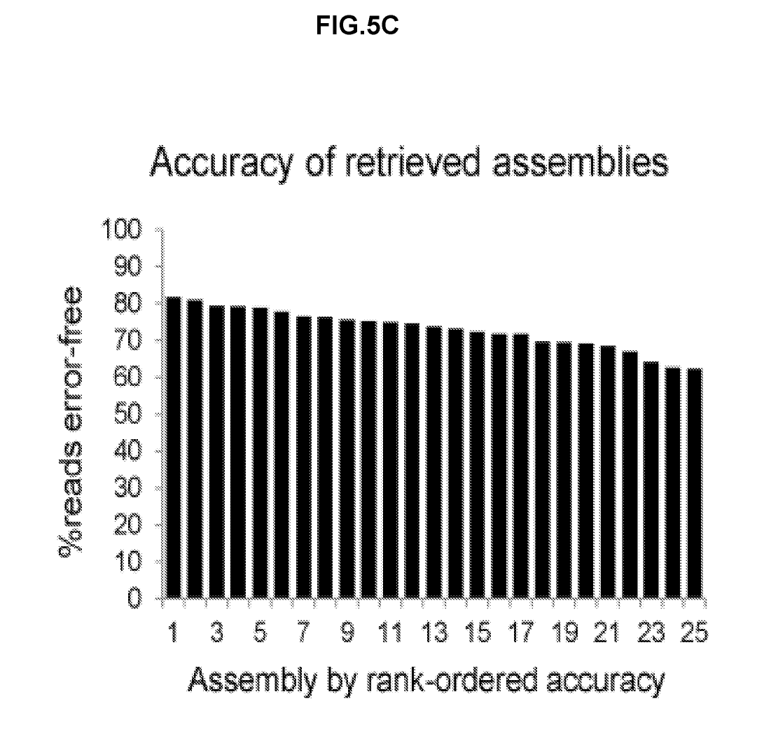

Oligonucleotide pools were sequenced and aligned to a reference of intended target sequences. For error analysis, one set of 250 targets was examined, each 237 bases long (set 5). Average nucleotide accuracy was calculated from bases with aligned reads having quality mapping score >20. A 98.68% average nucleotide accuracy of oligonucleotides was identified after amplification off the array. Since the assembly process relies on two priming sites and an overlap region, it was possible that assembly might intrinsically increase accuracy in these regions. Indeed, it was found that the average nucleotide accuracy of all aligning molecules in the 250-plex reaction was 99.02% (Poisson rate ratio 95% CI 1.36-1.38), showing the highest accuracy around the two priming sites and overlap region (FIG. 5A). In particular, the average nucleotide accuracy for the overlapping region increased from 98.53 to 99.44% (Poisson rate ratio 95% CI 2.64-2.77).

A significant increase in accuracy was observed at the nucleotide level (P˜4.9e-324), however a maximum of 37% perfect reads was observed in an assembled set. For downstream applications relying on accurate molecules, such as gene assembly, retrieving perfect assemblies from the assembled sets is of high interest. To do so, the Dial-Out PCR protocol was modified to incorporate a set of in-house static Dial-Out tags to allow for cost-efficient PCR retrieval of sequence-verified constructs.

Primers were designed that append M13F and M13R sequences during the assembly reaction for targets from sets 2 and 6 (each 250 targets). The assembled libraries were then tagged with the static Dial-Out tags, and sequenced for verification. The distribution of tag pairs was first analyzed, and it was found that 84.0% and 85.6% of all molecules in assembled and tagged sets 2 and 6 contained a unique, retrievable tag pair (out of 1.3 million reads for set 2 and 1.6 million reads for set 6) (FIG. 5B). 98.4% and 95.6% of targets had a sequence verified assembly with a unique tag pair.

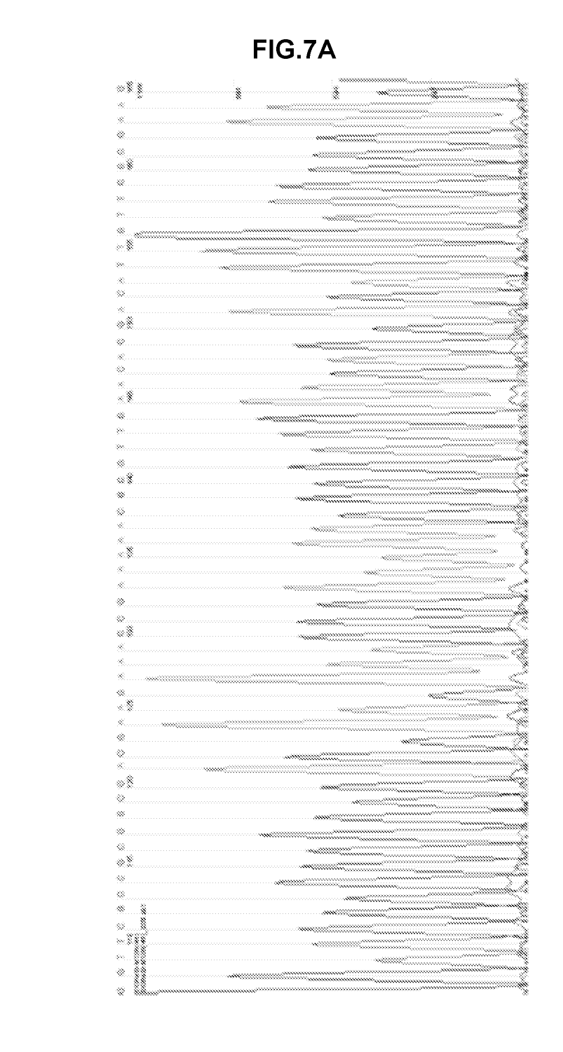

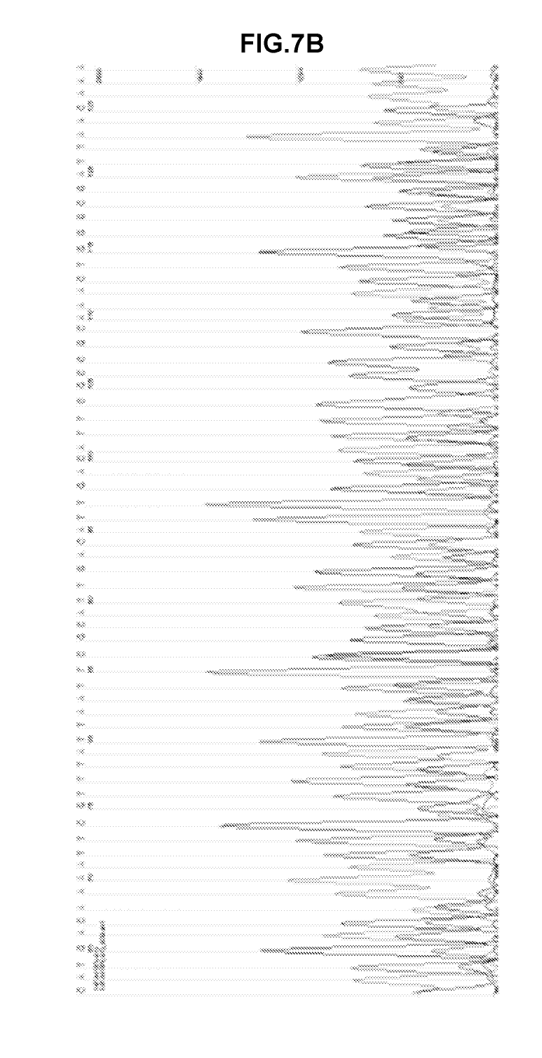

From set 2, 25 targets were chosen to retrieve, each of which was represented in at least 5 out of 1.3 million reads. All 25 targets amplified, and retrieval accuracy was evaluated by pooling all 25 retrieval reactions together and sequencing them with 1 million reads. All 25 targets were sequenced between 8,600 and 62,000 times, revealing error-free reads to the detection limit of Illumina sequencing chemistry, which is more quantitative than Sanger sequencing (FIG. 5C). A total of 78% of all sequencing reads aligned to one of the 25 targets. When aligned to all 2,271 potential targets, >99% of reads aligned, suggesting some background amplification of low abundance assemblies that was not observed in the sequencing, but that happen to share the same dial-out primer combinations. Consistent with this, Sanger sequencing revealed clean traces for 22 of the 25 targets, but high levels of noise for three traces (FIG. 7A and FIG. 7B).

### Example 4. Improved Uniformity and Yield

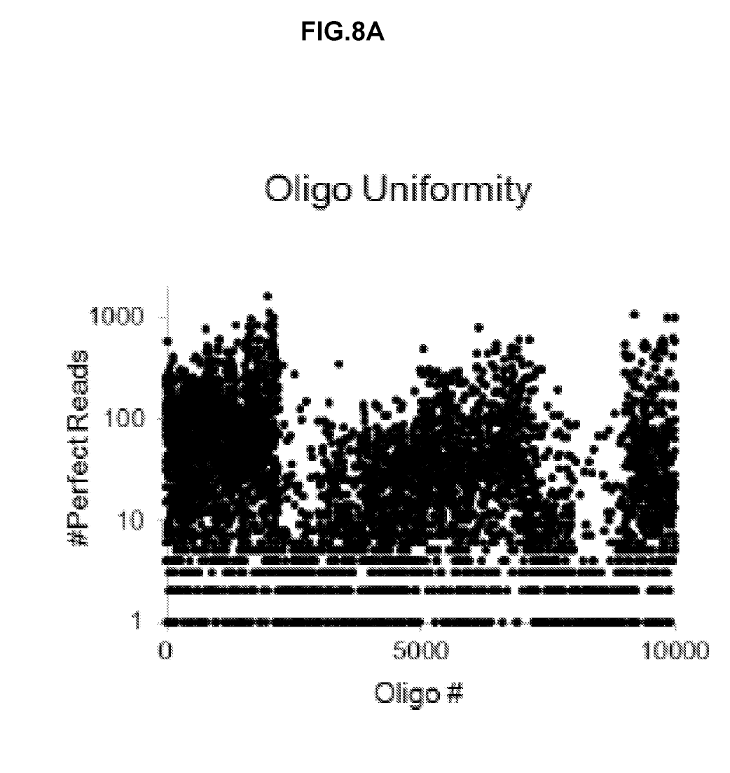

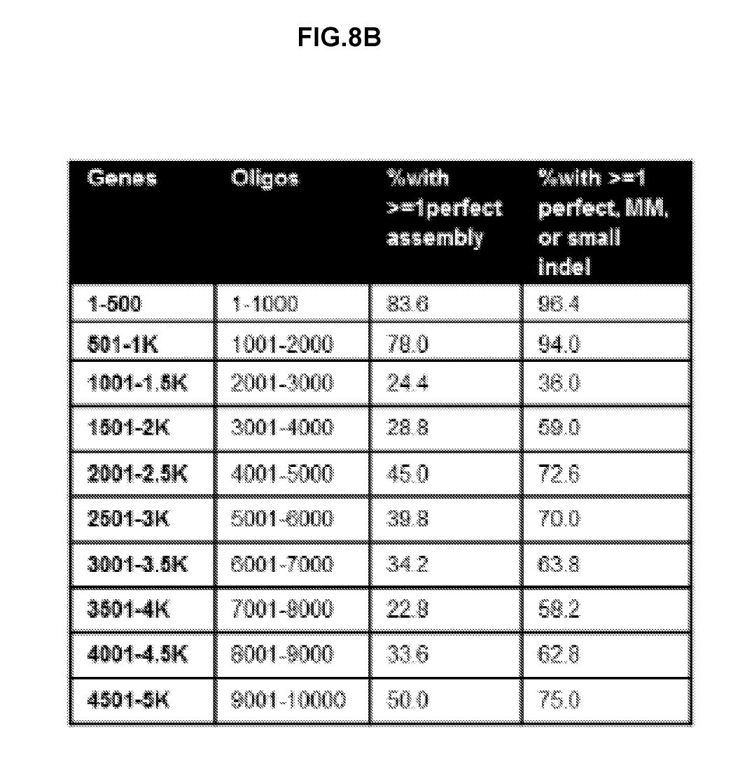

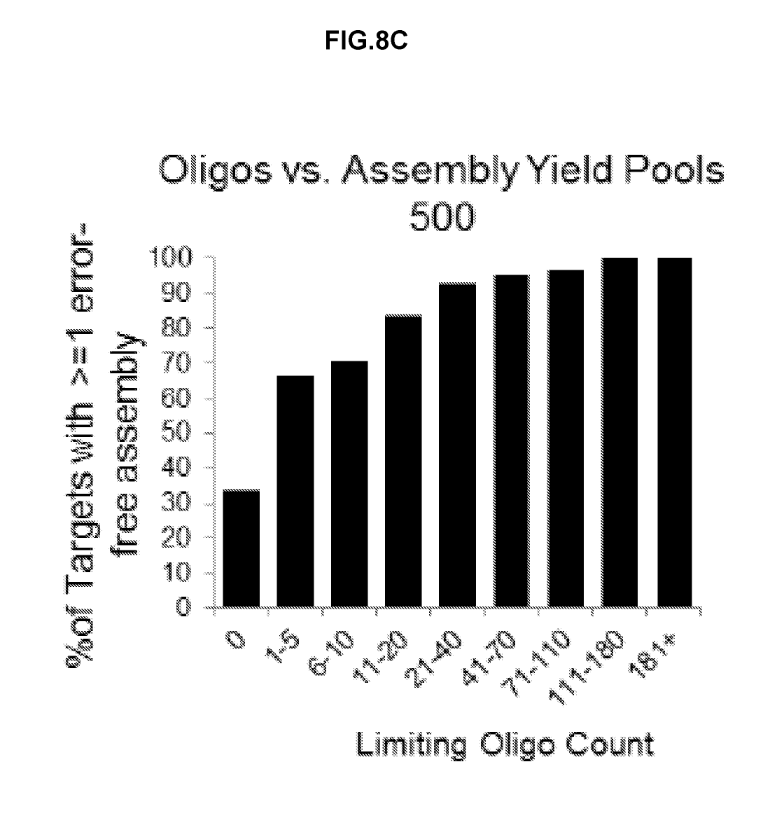

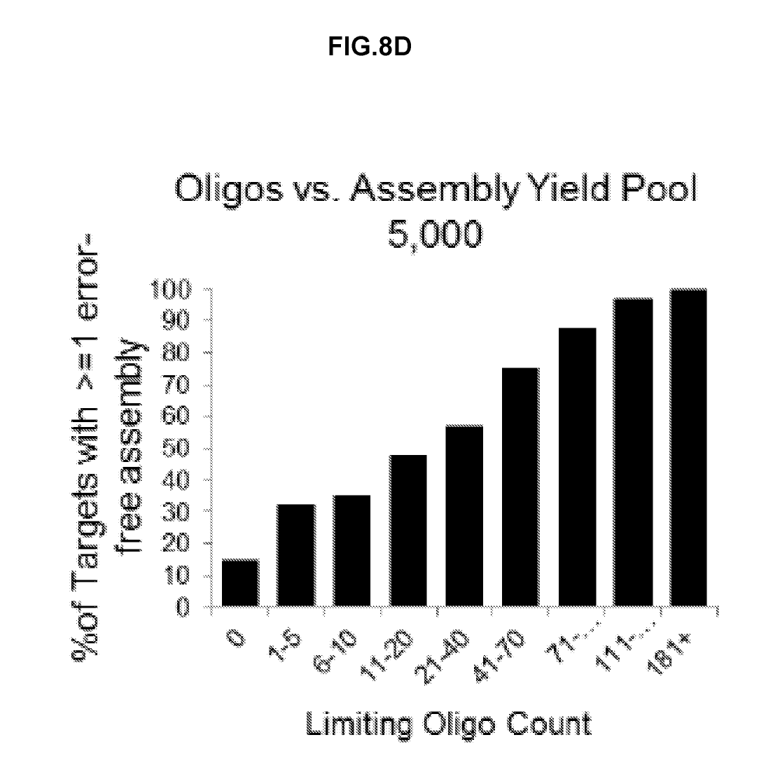

A potential limitation is the DNA synthesis error rate (e.g. mismatches and indels), moderate DNA assembly error rate (e.g. chimeras) and low uniformity. Low uniformity of input oligonucleotides impairs target uniformity in assembled sets. This is apparent in FIG. 4C, as well as a separate array in which oligonucleotides were not duplicated (FIG. 8). Increased yield and uniformity could occur if all oligonucleotides are duplicated during synthesis.

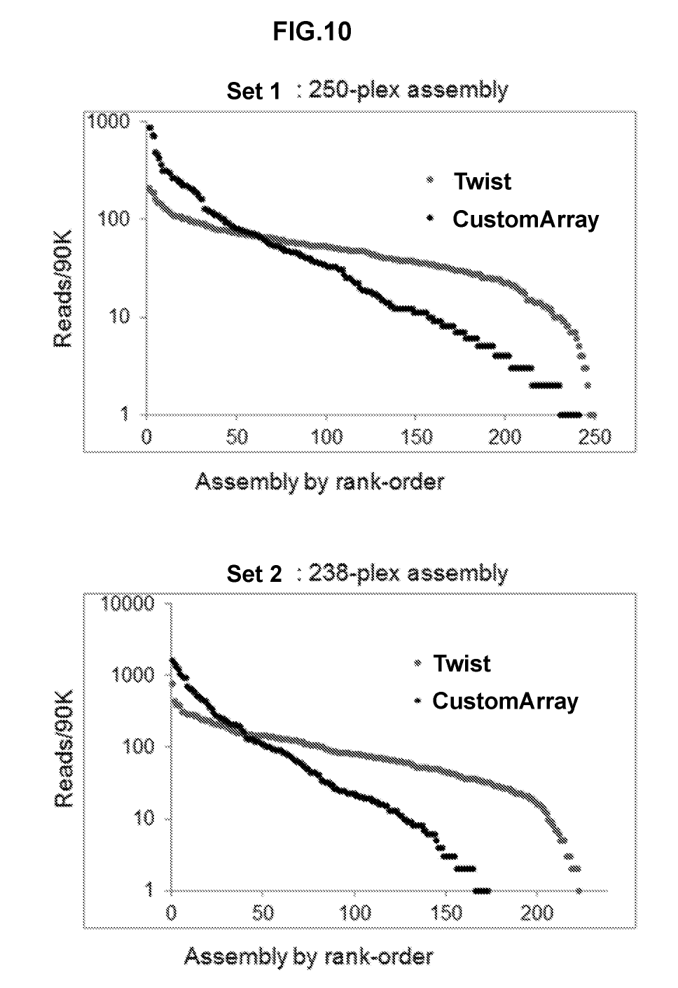

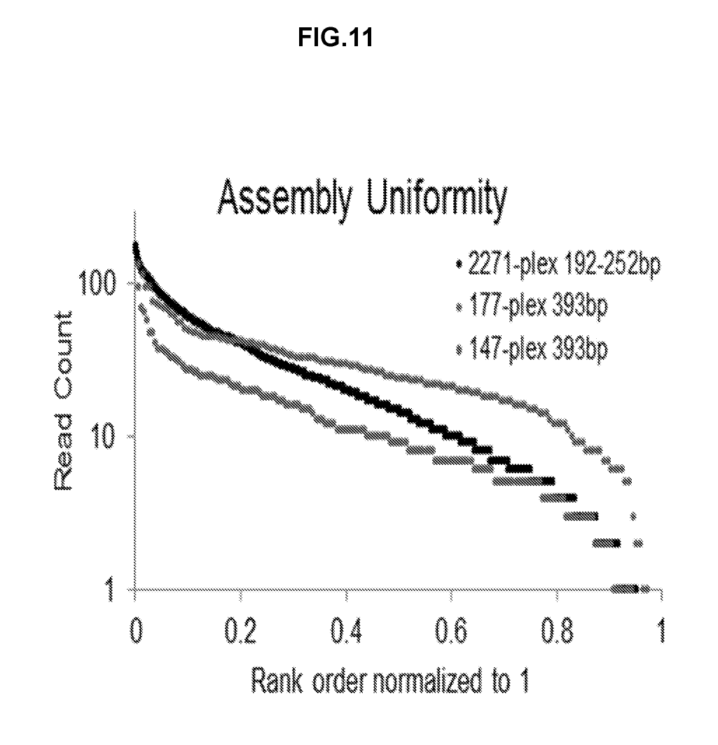

Higher uniformity, higher quality oligonucleotide pools synthesized by another manufacturer (Twist), which have a higher uniformity compared to CustomArray eliminated the need to duplicate all sequences on the array, and increased uniformity and yield in both cases (FIG. 10). Longer sequences can be assembled from longer oligonucleotides with minimal decrease in efficiency. Replicating the assembly of the entire 2,271-plex assembly with higher quality input oligonucleotides, improved the yield from 70.6% to >99%. Smaller sets of longer oligonucleotides (230 bp sequences) from a different vendor, Agilent, resulted in assembly of greater than 90% of 393 bp target sequences (FIG. 11).

### Example 5. Hierarchical Multiplex Pairwise Assembly

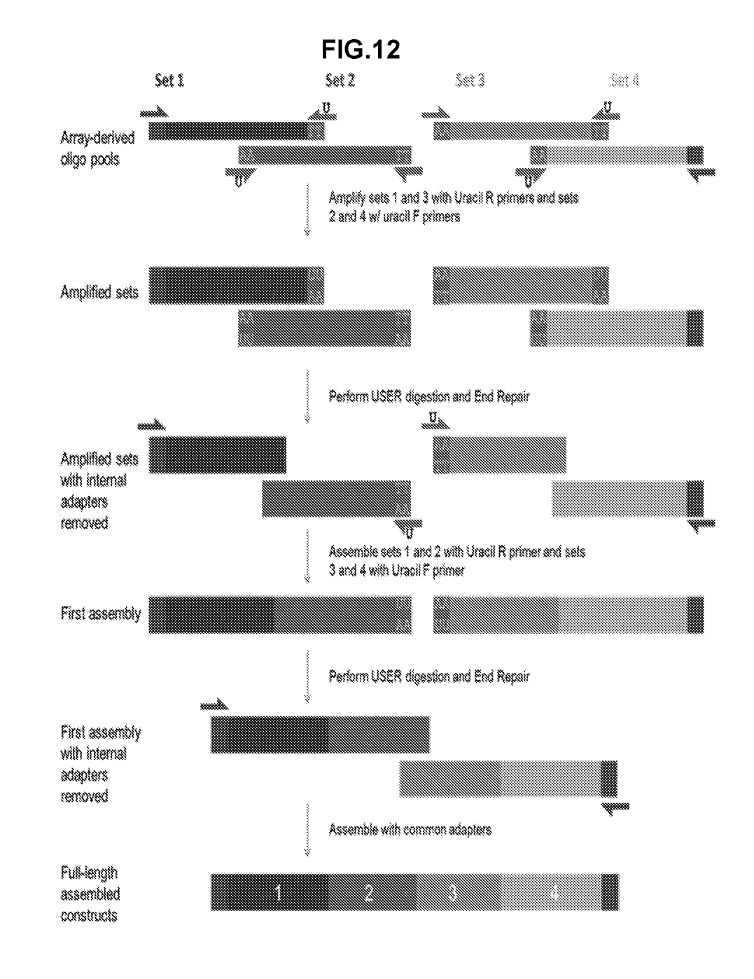

High-throughput functional screens would benefit from highly accurate and uniform assemblies. A general overview of hierarchical multiplex pairwise assembly is shown in FIG. 12. For gene assembly requiring very high accuracy, Dial-Out PCR was implemented to isolate perfect nucleotide sequences. For hierarchical assembly, yield is a concern, as every fragment must be represented in order to assemble larger constructs. In applications for hierarchical gene assembly, constructs should be assembled in smaller sets, as the methods disclosed herein are able to achieve yields up to >99% in sets of 250. However, in many applications, error-containing molecules can be filtered in the analysis stage, or may provide additional diversity for directed evolution. The spread in uniformity may also be accounted for with a post-hoc analysis by normalizing a post-selection sample to a pre-selection sample.

With the exception of chimeras, both the high error rate and lack of uniformity are due to the input reagents (see FIG. 10), and not the multiplex pairwise assembly method. The error profiles of the assembled sets match closely with the profile of the raw oligonucleotides (see FIG. 3). In fact, an increase in accuracy was observed at priming and assembly sites from the assembly protocol. Moreover, at least one error-free sequence was assembled for each target with high representation of both oligonucleotides, suggesting that much of the dropout and uniformity issues are due to poor uniformity in array synthesis. As shown above, using a higher-fidelity and more uniform array reduced these limitations.

The methods described herein can be inherently prone to producing chimeras. While these can be filtered out in most downstream applications, they may cause issues in more complex reactions by diluting the designed library. Chimeras can be minimized, to a maximum of 21.5%, by utilizing a custom script that examines all possible cross-hybridizations. In a separate experiment without the script, chimera rates as high as 42% were observed (FIG. 8). However, since the designs were different, a direct comparison of chimera rates can be compared.

Through Dial-Out PCR, the methods herein were able to retrieve error free assemblies for 25/25 targets. However, some background amplification was noticed, accounting for up to 22% of the sequenced pool. To reduce this noise in future methods, one could either increase the sequencing depth of the tagged pool or apply a more stringent filter for the number of times a construct was observed in the tagged pool.

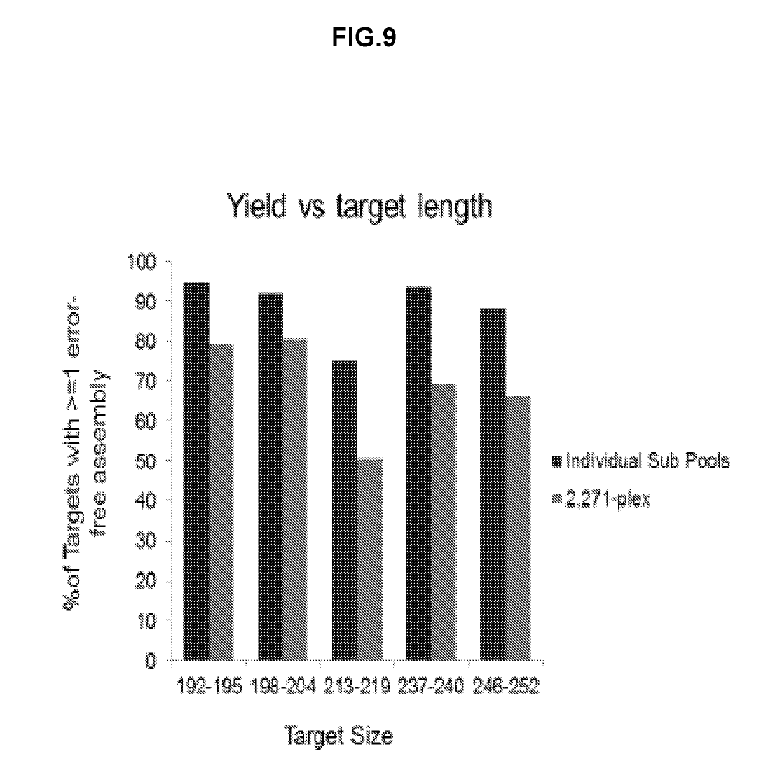

The method above was limited to synthesizing 252-mers as the maximum length of oligonucleotides because the input oligonucleotide pool was 160-mers (CustomArray). However, a decrease in yield was not observed with increasing target sizes from 191-252 bp (FIG. 9), target size can be increased by using longer oligonucleotide pools, for example, Agilent's 230-mers allow the assembly of 392-mers using the current methods (FIG. 11). As array technologies develop and longer oligonucleotides become available, the methods above can scale proportionately. Moreover, it is possible that the pairwise pools could be used for hierarchical assembly. This could occur directly after assembly, or after a round of multiplex Dial-Out PCR retrieval to reduce complexity and increase uniformity. It is also possible that the methods could be modified to assemble sets of three or more oligonucleotides instead of pairs, in a refined version of the shotgun synthesis technique described elsewhere (see Kim et al. (2012) Nucleic Acids Res., 40, e140.

The methods above for multiplex pairwise assembly of array derived DNA oligonucleotides provide methods for inexpensive, sequence-verified, oligonucleotide assembly from array synthesis. This is the first demonstration of assembly of thousands of array-derived oligonucleotides in multiplex, and use of a static set of PCR tags to retrieve sequence verified molecules. This protocol could be applicable for both complex library generation and gene synthesis. Creating a library of 3,118 such 200-mers is a surprisingly ˜38-fold less expensive than column-based synthesis methods (˜0.84 USD/target). Retrieving individual sequence-verified assemblies for each of the 3,118 is 17-fold less expensive with in-house Dial-Out tags and retrieval primers, and 4-fold less expensive including the one-time costs of the Dial-Out tag and retrieval primer libraries (Table 3). While column-based synthesis is limited to 200 bases, these methods synthesized 252-mers at 0.84 USD/target (0.0042 USD/base) with the similar efficiency as 200-mers (FIG. 9). With the advent of next-generation sequencing, high-throughput functional screens of DNA have shed light on the mechanisms of gene regulation and the classification of variants of uncertain significance. The ability to synthesize defined libraries at an unprecedented cost will allow researchers to address these questions using precisely designed sequences rather than relying on biased mutagenesis methods. Moreover, gene synthesis has contributed to novel pharmaceuticals and a better understanding of genome organization, and increasing the length of DNA assemblies that can be produced with low-cost, high complexity DNA synthesis will provide new opportunities for protein design and synthetic biology.

TABLE 3
Cost breakdown for 3,118 200-mers.

| | Multiplex Pairwise | Column-based |
|---|---|---|
| | Assembly | synthesis |
| Raw oligo cost | $2,400 | $99,776 |
| Oligo Pool amplification | $24 | — |
| with Kapa HiFi Uracil+ | | |
| USER treatment + End | $36 | — |
| Repair | | |
| Assembly PCR | $12 | — |
| Sequence Verification | $150 | — |
| Total Cost | $2,622 | $99,776 |
| Dial-Out Tag Library- | $1,800 | — |
| one-time cost | | |
| Retrieval Primer Library- | $17,118 | — |
| one-time cost | | |
| Dial-Out: Total one-time | $18,918 | — |
| cost | | |
| Dial-Out Tagging and | $150 | — |
| Sequencing | | |
| Dial-Out Retrieval | $3,118 | — |
| Total Cost with Dial-Out | $5,890 | — |
| Retrieval | | |

Raw oligo cost for multiplex pairwise assembly is based on dupicating all oligonucleotides and filling one 12,472-array from CustomArray with 160mers. Column-based oligonucleotide cost is based on IDT price of 384-well sub-nanomole plates. Note that IDT cannot synthesis oligonucleotides longer than 200 bp by this method, whereas the methods described herein demonstrate the synthesis of 252-mers. The rest of the steps for multiplex pairwise assembly are based on separating targets into six pools. Sequencing costs are based on a MiSeq v2 300 cycle spike-in (2 million reads). For Dial-Out Tagging, there is a one-time cost of the tag and PCR retrieval libraries. The total cost with Dial-Out Retrieval does not include the one-time cost.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

While the invention has been particularly shown and described with reference to an aspect and various alternate aspects, it will be understood by persons skilled in the relevant art that various changes in form and details can be made therein without departing from the spirit and scope of the invention. The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize

All references, issued patents and patent applications cited within the body of the instant specification are hereby incorporated by reference in their entirety, for all purposes. Aspects of the disclosure can be modified, if necessary, to employ the systems, functions, and concepts of the above references and application to provide yet further embodiments of the disclosure. These and other changes can be made to the disclosure in light of the detailed description.

### REFERENCES

ALLAWI and SANTA-LUCIA, (1997) Thermodynamics and NMR of internal G.T mismatches in DNA. Biochemistry, 36, 10581-10594.
BANG and CHURCH (2008). Gene synthesis by circular assembly amplification. Nat. Methods, 5, 37-39.
BEAUCAGE and CARUTHERS, (1981) Deoxynucleoside phosphoramidites-a new class of key intermediates for deoxynucleotide synthesis. Tetrahedron Lett., 22, 1859-1862.
BINKOWSKI et al., (2005) Correcting errors in synthetic DNA through consensus shuffling. Nucleic Acids Res., 33, e55.
BLANCHARD et al., (1996) High-density oligonucleotide arrays. Biosens. Bioelectron., 11, 687-690.
BOROVKOV et al., (2010) High-quality gene assembly directly from unpurified mixtures of microarray-synthesized oligonucleotides. Nucleic Acids Res., 38, el80.
CARR et al., (2004) Protein-mediated error correction for de novo DNA synthesis. Nucleic Acids Res., 32, e162.
DORMITZER et al., (2013) Synthetic generation of influenza vaccine viruses for rapid response to pandemics. Sci. Transl. Med., 5, 185ra68.
FINDLAY et al., (2014) Saturation editing of genomic regions by multiplex homology-directed repair. Nature, 513, 120-123.
FUHRMANN et al., (2005) Removal of mismatched bases from synthetic genes by enzymatic mismatch cleavage. Nucleic Acids Res., 33, e58.
GHINDILIS et al., (2007) Combimatrix oligonucleotide arrays: genotyping and gene expression assays employing electrochemical detection. Biosens. Bioelectron., 22, 1853-1860.
HUGHES et al., (2001) Expression profiling using microarrays fabricated by an ink-jet oligonucleotide synthesizer. Nat. Biotechnol., 19, 342-347.
KIM et al., (2012) ‘Shotgun DNA synthesis’ for the high-throughput construction of large DNA molecules. Nucleic Acids Res., 40, e140.
KONG et al., (2007) Parallel gene synthesis in a microfluidic device. Nucleic Acids Res., 25, e61.
KOSURI and CHURCH, (2014) Large-scale de novo DNA synthesis: technologies and applications. Nat. Methods, 11, 499-507.
KOSURI et al., (2010) Scalable gene synthesis by selective amplification of DNA pools from high-fidelity microchips. Nat. Biotechnol., 28, 1295-1299.
KRISTIANSSON et al., (2009) Evolutionary forces act on promoter length: identification of enriched cis-regulatory elements. Mol. Biol. Evol., 26, 1299-1307.
LEVINE and TJIAN, (2003) Transcription regulation and animal diversity. Nature, 424, 147-151.
LINSHIZ et al., (2008) Recursive construction of perfect DNA molecules from imperfect oligonucleotides. Mol. Syst. Biol., 4, 191.
MARKHAM and ZUKER, (2008) UNAFold: software for nucleic acid folding and hybridization. Methods Mol. Biol., 453, 3-31.
MATZAS et al., (2010) High-fidelity gene synthesis by retrieval of sequence-verified DNA identified using high-throughput pyrosequencing. Nat. Biotechnol., 28, 1291-1294.
MELNIKOV et al., (2012) Systematic dissection and optimization of inducible enhancers in Biotechnol., 30, 271-277.
NGUYEN-DUMONT et al., (2013) A high-plex PCR approach for massively parallel sequencing. Biotechniques, 55, 69-74.
PATWARDHAN et al., (2009) High-resolution analysis of DNA regulatory elements by synthetic saturation mutagenesis. Nat. Biotechnol., 27, 173-1175.
QUAN et al., (2011) Parallel on-chip gene synthesis and application to optimization of protein expression. Nat. Biotechnol., 29, 449-452.
SAAEM et al., (2010) In situ synthesis of DNA microarray on functionalized cyclic olefin copolymer substrate. ACS Appl. Mater. Interfaces, 2, 491-497.
SAMBROOK et al., (1989) Molecular Cloning: A Laboratory Manual. Cold Spring Harbor Laboratory Press, Cold Spring Harbor, N.Y.
SCHLABACH et al., (2010) Synthetic design of strong promoters. Proc. Natl. Acad. Sci. U.S.A., 107, 2538-2543.
SCHWARTZ et al., (2012) Accurate gene synthesis with tag-directed retrieval of sequence-verified DNA molecules. Nat. Methods, 9, 913-915.
SHARON et al., (2012) Inferring gene regulatory logic from high-throughput measurements of thousands of systematically designed promoters. Nat. Biotechnol., 30, 521-530.
SMITH et al., (2013) Massively parallel decoding of mammalian regulatory sequences supports a flexible organizational model. Nat. Genet., 45, 1021-1028.
SMITH and MODRICH (1997) Removal of polymerase-produced mutant sequences from PCR products. Proc. Natl. Acad. Sci. U.S.A., 94, 6847-6850.
TIAN et al., (2004) Accurate multiplex gene synthesis from programmable DNA microarrays. Nature, 432, 1050-1054.
WAN et al., (2014) Error removal in microchip-synthesized DNA using immobilized MutS. Nucleic Acids Res., 42, e102.
XU and NUSSINOV, (1998) Favorable domain size in proteins. Fold. Des., 3, 11-17.
YOUNG and DONG, (2004) Two-step total gene synthesis method. Nucleic Acids Res., 32, e59.
ZHANG et al., (2014). PEAR: a fast and accurate Illumina Paired-End read merger. Bioinformatics, 30, 614-620.
ZHOU et al., (2004) Microfluidic PicoArray synthesis of oligodeoxynucleotides and simulataneous assembling of multiple DNA sequences. Nucleic Acids Res., 32, 5409-5417.

1: A method for assembly of one or more double-stranded polynucleotides, the method comprising:
(a) amplifying a first plurality of single-stranded overlapping oligonucleotides, wherein the first plurality of single-stranded overlapping oligonucleotides comprises:
(i) overlapping regions with homology capable of annealing to produce one or more double-stranded polynucleotides, and
(ii) at least one common primer binding site in each single-stranded overlapping oligonucleotide;
(b) assembling one or more double-stranded polynucleotides, wherein the assembling comprises denaturing, annealing and extending the first plurality of single-stranded overlapping oligonucleotides to generate the one or more double-stranded polynucleotides.

2: The method of claim 1, further comprising:
(c) tagging the one or more double-stranded polynucleotides, wherein the tagging comprises amplifying the one or more double-stranded oligonucleotides using a pair of tagging primers to generate one or more tagged double-stranded polynucleotides, wherein each tagging primer in the pair of tagging primers comprises:
(i) a first segment comprising a unique flanking sequence, and
(ii) a second segment comprising a seed sequence;
(d) sequencing the one or more tagged double-stranded polynucleotides, wherein the sequencing comprises binding of the seed sequence to a sequencing platform and performing a sequencing reaction to identify one or more sequence verified polynucleotides; and
(e) retrieving the one or more sequence verified polynucleotides, wherein the retrieving comprises base-pairing a complementary primer to the first segment of at least one tagging primer in the one or more sequence verified polynucleotides and, under conditions suitable and in the presence of suitable reagents, amplifying the sequence verified polynucleotides to produce one or more verified polynucleotides; or
(c) phenotypic selection of functional polypeptides, wherein the phenotypic selection comprises of one or more of yeast display, phage display, mRNA display, ribosome display, mammalian cell display, bacterial cell display, emulsion-based protein selection, functional complementation of a portion of a genome, or other selection methods known to experts in the field of polypeptide evolution.

3: The method of claim 1, wherein the one or more double-stranded polynucleotides comprises at least 100 to 2,000 double-stranded polynucleotides.

4: The method of claim 2, further comprising step-wise assembly of two or more of the double-stranded or verified polynucleotides into an assembled polynucleotide product, wherein the two or more double-stranded or verified polynucleotides have overlapping regions with homology capable of annealing and at least one common primer binding site in each of the double-stranded or verified polynucleotides; and
(f) combining the two more double-stranded or verified polynucleotides under conditions suitable for annealing the overlapping regions with homology and in the presence of suitable reagents for assembling an initial desired polynucleotide product by extension of the double-stranded or verified polynucleotides to produce the initial desired polynucleotide product; and
(g) combining the initial desired polynucleotide product and a next double-stranded or verified polynucleotide, wherein the initial desired polynucleotide product and the next double-stranded or verified polynucleotide have overlapping regions with homology capable of annealing and at least one common primer binding site in each of the double-stranded or verified polynucleotides, and assembling the initial desired polynucleotide product and the next double-stranded or verified polynucleotide in the presence of suitable reagents for assembling the assembled polynucleotide product by extension of the initial desired polynucleotide product and the next double-stranded or verified polynucleotide; and
(h) reiteratively repeating (g) to step-wise add additional next double-stranded or verified polynucleotides to the initial desired polynucleotide product to produce the assembled polynucleotide product.

5: The method of claim 2, further comprising hierarchical assembly of two or more of the double-stranded or verified polynucleotides into an assembled polynucleotide product, wherein the two or more double-stranded or verified polynucleotides have overlapping regions with homology capable of annealing and at least one common primer binding site in each of the double-stranded or verified polynucleotides; and
(f) combining the two double-stranded or verified polynucleotides under conditions suitable for annealing the overlapping regions with homology and in the presence of suitable reagents for assembling a first desired polynucleotide product by extension of the double-stranded or verified polynucleotides to produce the first desired polynucleotide product; and
(g) repeating (f) with another two double-stranded or verified polynucleotides to produce a second desired polynucleotide product;
(e) combining the first desired polynucleotide product and the second desired polynucleotide product, wherein the first desired polynucleotide product and the second desired polynucleotide product have overlapping regions with homology capable of annealing and at least one common primer binding site in each of the first and the second desired polynucleotide products, and assembling the first desired polynucleotide product and the second desired polynucleotide product in the presence of suitable reagents for assembling the assembled polynucleotide product by extension of the first desired polynucleotide product and the second desired polynucleotide product; and
(h) repeating (f), (g) and (e) to hierarchically assemble pairs of desired polynucleotides to produce the assembled polynucleotide product.

6: The method of claim 1, wherein the nucleotide sequence of each of the oligonucleotides in the first plurality of single-stranded overlapping oligonucleotides is a predefined sequence.

7. (canceled)

8. (canceled)

9: The method of claim 1, wherein the first plurality of single-stranded overlapping oligonucleotides is derived from an array.

10. (canceled)

11: The method of claim 1, wherein the first plurality of single-stranded overlapping oligonucleotides is amplified from the array using a common primer and a set-specific primer.

12: The method of claim 1, wherein the first plurality of single-stranded overlapping oligonucleotides comprises at least one uracil-containing primer region.

13: The method of claim 11, wherein the set-specific primer is also a uracil-containing primer.

14. (canceled)

15. (canceled)

16: The method of claim 12, wherein the uracil-containing primer region is removed from the first plurality of single-stranded overlapping oligonucleotides by contacting the oligonucleotides with uracil DNA glycosylase (UDG) and a DNA glycosylase-lyase endonuclease VIII.

17: The method of claim 1, wherein the overlapping regions have a melting temperature (Tm) that is greater than 56° C.

18. (canceled)

19: The method of claim 1, wherein assembly of the one or more double-stranded oligonucleotides comprises between 5-30 cycles of denaturing, annealing and extending.

20. (canceled)

21: The method of claim 1, wherein assembly of the double-stranded or verified polynucleotides occurs in sets.

22: The method of claim 21, wherein the sets range from approximately 100 to 2,275 double-stranded or verified polynucleotides.

23. (canceled)

24. (canceled)

25. (canceled)

26: The method of claim 1, wherein the method comprises assembling more than 2,000 of the double-stranded or verified polynucleotides, and wherein the double-stranded or verified polynucleotides are assembled with >50% accuracy.

27: The method of claim 2, wherein the unique flanking sequence of the tagging primers comprises a 13 nucleotide sequence with the following properties:
(a) no more than 5 consecutive nucleotide residues of homoguanine or homocysteine;
(b) no more than 8 consecutive nucleotide residues of homoadenine or homothymine; and
(c) a guanine-cysteine (GC) content between 45% and 65%.

28: The method of claim 2, wherein the seed sequence of the tagging primers comprise a sequence of 15-25 nucleotides capable of binding of the seed sequence to a sequencing platform and performing a sequencing reaction.

29. (canceled)

30: The method of claim 1, wherein the first plurality of single-stranded oligonucleotides or the double-stranded polynucleotides are at least about 100 to 400 nucleotides in length.

31. (canceled)

32: The method of claim 1, wherein the assembled polynucleotide product is at least about 250 to 300,000 nucleotides in length.

33. (canceled)

34. (canceled)
